(19) United States
(12) Patent Application Publication
(10) Pub. No.: US 2024/0384297 A1
(43) Pub. Date: Nov. 21, 2024

(54) GENE THERAPY CONSTRUCT FOR TREATING POMPE DISEASE, PHARMACEUTICAL COMPOSITION, AND METHOD

(71) Applicant: BEIJING GENECRADLE PHARMACEUTICAL CO., LTD., Beijing (CN)

(72) Inventor: Xiaobing WU, Beijing (CN)

(73) Assignee: BEIJING GENECRADLE PHARMACEUTICAL CO., LTD., Beijing (CN)

(21) Appl. No.: 18/690,068

(22) PCT Filed: May 27, 2022
(86) PCT No.: PCT/CN2022/095637

§ 371 Date: Mar. 7, 2024

(30) Foreign Application Priority Data
Sep. 8, 2021 (CN) 202111051212.9

(51) Int. Cl. C12N 15/86; A61K 38/47; A61K 48/00; C12N 9/24; C12N 15/113

(52) U.S. Cl. CPC C12N 15/86; A61K 38/47; A61K 48/005; A61K 48/0075; C12N 9/2402; C12N 15/113; C12Y 302/0102; C12N 2310/141; C12N 2750/14143; C12N 2750/14151; C12N 2830/50

(57) ABSTRACT

Provided are a constitutive promoter CAR-Mut, an expression construct comprising the promoter and a GAA coding nucleotide sequence functionally linked thereto, a recombinant vector and a host cell. Also provided are a composition and method for delivering a GAA coding polynucleotide to a mammalian cell or an individual using the recombinant vector, and for treating a subject with Pompe disease or acid glucosidase deficiency.

### TECHNICAL FIELD

The present invention relates to gene therapy. More particularly, the present invention relates to constructs in gene therapy for Pompe disease, as well as pharmaceutical compositions comprising the constructs and methods for treating Pompe disease.

### BACKGROUND ART

### Gene Therapy

The first authorized gene therapy research came into being in 1989. After more than three decades of development, gene therapy has achieved a milestone breakthrough and entered a new era. Gene therapy has made a great progress in treating previously incurable genetic diseases. Several gene therapy drugs have been approved by the FDA/EMA for clinical use. More gene therapy drugs for more genetic diseases, such as neuromuscular diseases and hemophilia, are also expected to obtain further approvals in the future. In addition, gene therapy is now being widely used in research for the treatment of tumors, infectious diseases, cardiovascular diseases and autoimmune diseases.

A key to gene therapy drugs is to adopt a suitable vector material to deliver an exogenous gene to a recipient cell, and achieve the purpose of disease treatment through transcription and expression of the exogenous gene. Currently, the commonly used gene therapy vectors mainly include virus and non-virus types. Virus vectors are widely used because of their natural characteristics capable of efficiently introducing exogenous genes into recipient cells. Among virus vectors, adeno-associated virus (AAV) vectors have become one of the most active vectors for in vivo gene therapy because of their good safety and efficient transduction to a variety of target tissues.

As an important element in a gene engineering expression vector, a promoter can largely determine the expression efficiency and the tissue expression profile of a cloned gene. Therefore, in the field of gene therapy, in order to meet the needs of gene therapy, it is often necessary to create a new promoter that satisfies the requirements based on specific therapeutic purposes. At the same time, there is also an objective need to provide a diverse selection of promoters in this field.

The capacity of virus vectors as transgene delivery vehicles to accommodate genes is limited. In the case of conventional single-stranded AAV virus vectors (ssAAV), the total packaging capacity is about 4.8 kb. In the case of double stranded self-complementary AAV virus vectors (scAAV), the total packaging capacity is about half of that in ssAAV, i.e. about 2.5 kb. Therefore, it is particularly important to choose an appropriate combination of vector genetic elements to accommodate the size of the genetic construct, while ensuring a gene of interest is expressed at the desired level in the desired tissue (or multiple tissues).

In some cases, constitutive expression of a transgene in all or most cell types is desired, for example, when a disease or a disorder being treated involves multiple tissues. Some constitutive promoters have been provided in the art, such as human elongation factor 1 promoter, cytomegalovirus CMV promoter, chicken actin CBA promoter, and the synthetic CAG promoter comprising a CMV enhancer. However, the use effectiveness of a constitutive promoter often varies depending on the specific disease or diseased tissue to which the constitutive promoter is applied, the mode of administration, and other factors, and in some instances, a constitutive promoter may bring about a higher level of drug immunogenicity and/or animal toxicity, thereby limiting its use in a drug construct for gene therapy. Accordingly, there is an ongoing need for gene therapy constructs that are both more efficient in transducing disease-related tissues and safer.

### Pompe Disease

Pompe disease, also known as acid alpha-glucosidase deficiency or glycogen storage disease type II (GSDII), is a systemic lysosomal storage disease that primarily involves the muscles but also affects the central nervous system. In affected individuals, a lack of functional acid alpha-glucosidase (GAA) in lysosomes results in the inability of glycogen to be converted to glucose and utilized, rendering the accumulation of glycogen in the lysosomes of the cells of the patients, especially of the cells in the peripheral organs and tissues, such as skeletal and cardiac muscles, and in the central nervous system, including the brain and the spinal cord, which leads to the disease. An enzymatic activity testing can be done to measure the activity of alpha-glucosidase for diagnosis of Pompe disease.

According to the age of onset and severity, Pompe disease can be categorized as: infantile-onset and late-onset. Individuals with infantile-onset Pompe disease (IOPD) have a very low residual GAA enzyme activity, exhibit more severe symptoms such as dyspnea, generalized muscle weakness and cardiorespiratory failure, and are often lethal. Individuals with childhood-to-adult-onset Pompe disease have a slower progression of the disease due to a higher residual GAA enzyme activity. This milder form of Pompe disease is also known as late-onset Pompe disease (LOPD). Heart muscle defects are often absent in individuals with LOPD, but muscle weakness may lead to severe respiratory problems and respiratory failure.

The only currently approved treatment for Pompe disease is enzyme replacement therapy (ERT). ERT offers the advantage of consistently improving abnormal cardiac function and preventing heart failure. However, ERT exhibits limitations for affected skeletal muscle and CNS system. Individual patients treated with ERT can have widely varying skeletal muscle responses. One possible factor contributing to this variability in response is believed to be the development of high-titer antidrug antibodies. Studies in animals and humans have suggested that antibodies developed against GAA enzyme can reduce the efficacy of ERT. In addition, ERT drugs are unable to cross the blood-brain barrier to treat CNS lesions and affected respiratory motor neurons. Severe progressive neurodegeneration has been reported in infant individuals receiving ERT. Brain MRI studies in long-term survivors of ERT treatment have also revealed slowly progressive white matter damage. Another limitation of ERT is the complete absence of glycogen clearance, or inadequate glycogen clearance, in certain tissue types, such as smooth muscles in the vasculature, eyes, gastrointestinal tract, and respiratory system.

Pathologically, Pompe disease, an autosomal recessive monogenic disorder, is caused by pathologic mutations in the acid alpha-glucosidase (GAA) gene (including various nonsense and missense mutations that result in the loss or reduction of GAA enzyme activity). Therefore, as an alternative or complement to ERT, a gene therapy approach is proposed to control GAA gene defects in individual patients.

Darin J Falk et al. (2013, Intrapleural Administration of AAV9 Improves Neural and Cardiorespiratory Function in Pompe Disease, doi: 10.1038/mt.2013.96) used AAV9 carrying a recombinant GAA gene under the control of a constitutive CMV promoter and a tissue-specific promoter DES, and treated Pompe disease mice by intrapleural injection of the AAV9. The results showed that there was an increase in GAA enzyme activity in the heart, but almost no GAA enzyme activity was detected in the liver.

Enyu Deng et al. (MOLECULAR THERAPY Vol. 5, No. 4, 2002; doi: 10.1006/mthe.2002.0563) used an AAV vector carrying a constitutive CMV promoter and a recombinant GAA gene (Ad CMV-GAA) to treated Pompe disease in mice by intravenous injection. The results indicated that transient high levels of GAA were observed in plasma following the injection of the recombinant AAV. However, the CMV promoter quickly shut down a few days after the vector injection, and a rapid development of anti-GAA antibodies was induced by the AAV gene treatment. This resulted in a swift decline in plasma GAA to a completely undetectable level.

To address the immune response induced by AAV vector drugs, Sang-oh Han et al. (Molecular Therapy: Methods & Clinical Development Vol. 4 Mar. 2017, http://dx.doi.org/10.1016/j.omtm.2016.12.010.) proposed using a liver-specific promoter (LSP) instead of a constitutive promoter and incorporating a liver-targeted AAV2/8 to develop an AAV vector named AAV2/8-LSPhGAA for treating GAA knockout (KO) mice. The study evaluated the efficacies of AAV2/8-LSPhGAA at three lower doses, when used alone or in combination with ERT. The results showed that AAV vectors carrying hGAA under the control of a constitutive promoter of CMV enhancer/CB promoter did not induce immune tolerance, while AAV8-LSP-hGAA utilizing the liver-specific promoter LSP could be beneficial in suppressing anti-GAA antibody responses. However, AAV2/8 targets the liver, and the proteins produced therefrom are unable to cross the blood-brain barrier to reach the central nervous system and alleviate the central nervous system involvement in Pompe disease. See also WO2009075815A1.

Allison M. Keeler et al. and Jeong-A Lim et al. proposed to improve the efficacy of AAV gene therapy on the affected CNS by modifying the viral capsid. In a study by Allison M. Keeler (Systemic Delivery of AAVB1-GAA Clears Glycogen and Prolongs Survival in a Mouse Model of Pompe Disease, HUMAN GENE THERAPY, VOLUME 30 NUMBER 1, DOI: 10.1089/hum.2018.016), an adeno-associated virus (AAV) vector AAVB1-DES-h GAA was constructed by using AAVB1 having a high affinity for muscle and CNS, along with AAV9-DES-h GAA vector as control. After injection of the vectors via the tail vein of GAA KO mice in which the GAA gene was knocked out, both vectors efficiently transduced the heart, leading to glycogen clearance, and the transductions of the diaphragm and central nervous system were observed on tissue sections. However, only AAVB1 treated mice exhibited stable weight gain and restoration of limb strength. In addition, limited by the tissue specificity and weaker expression level of the DES promoter, the hepatic GAA levels in AAV-treated animals were significantly lower than that in the wild type, and the GAA activities in the trachea, medulla, cervical, thoracic, and lumbar spinal cord in both AAV-treated groups were below the limit of detection of the enzyme assay.

In a study by Jeong-A Lim et al. (Molecular Therapy: Methods & Clinical Development Vol. 12 Mar. 2019; https://doi.org/10.1016/j.omtm.2019.01.006.), an AAV virus vector was constructed by using a viral capsid PHP.B, and the glycogen content was reduced to the wild-type level in brain and heart and significantly reduced in skeletal muscle after a single intravenous injection of AAV-PHP.B-CB-GAA into 2-week-old GAA KO mice. The transduction efficiency of PHP.B-CB-hGAA was sufficient to prevent the accumulation of glycogen in the brain of GAA KO mice and to rescue the associated neural phenotype. Unfortunately, however, this unusually high CNS targeting of the PHP.B capsid was limited to a specific transgenic mouse model.

Gene therapy using an AAV vector (rAAV1-CMV-hGAA) delivered intra-diaphragmatically was clinically tested in pediatric patients with Pompe disease. The clinical trial confirmed the safety of the AAV vector; however, the clinical outcome was not significant and an anti-capsid and anti-transgene antibody responses were observed in all patients without immunomodulators received. (Giuseppe Vita, 2019, https://doi.org/10.1007/s10072-019-03764-z).

Thus, there remains a continuing need in the field of gene therapy for Pompe disease, to provide new therapeutic vectors and drugs to achieve effective transduction of disease-associated tissues and amelioration of lesions, as well as a reduced anti-drug immune response.

### SUMMARY OF INVENTION

The present inventors have proposed, after intensive research, a novel synthetic constitutive promoter, a novel AAV virus vector based on said promoter and uses thereof, which when administered intravenously, alleviate the burden of Pompe disease in the central nervous system, address peripheral organ involvement, and exhibit low drug immunogenicity.

Accordingly, in one aspect, the present invention provides a mutant promoter comprising a polynucleotide of SEQ ID NO: 4, a polynucleotide having at least 95% identity to SEQ ID NO: 4, or a polynucleotide having one or several nucleotide alterations relative to SEQ ID NO: 4, wherein the polynucleotide bears a mutation at positions 562-572 of SEQ ID NO:4, preferably a mutation from T to C, G or A, particularly a T to C mutation, at position 568. Compared with a reference promoter without the mutation, the mutant promoter according to the present invention increases the expression of a gene of interest functionally linked thereto, especially the expression in mammalian cells or tissues. The strong promoter activity of the mutant promoter according to the present invention makes it particularly suitable for therapeutic use in Pompe disease.

In a further aspect, the present invention provides expression constructs, vectors, host cells comprising the mutant promoter of the present invention, and pharmaceutical compositions thereof.

In a further aspect, the present invention provides recombinant AAV virus vectors comprising the mutant promoter of the present invention and a polynucleotide encoding acid alpha-glucosidase GAA. The virus vectors of the present invention may be ssAAV or scAAV virus vectors. Preferably, the virus vectors of the present invention comprise AAV capsid proteins targeting muscle and/or nervous system, such as capsid proteins of AAV9 serotype.

In a further aspect, the present invention provides methods for using the recombinant virus vectors of the present invention in subjects with Pompe disease or with acid glucosidase deficiency for the treatment or prevention of the disease or the deficiency, and also provides uses of the recombinant virus vectors of the present invention in the manufacture of a medicament for the prevention or treatment of the disease or the deficiency. In preferred embodiments, the methods of the present invention increase the level of GAA enzyme activity and a decrease in glycogen storage in peripheral tissues and central nervous system tissues of the subjects. The methods of the present invention advantageously alleviate the burden of Pompe disease in the central nervous system, address peripheral organ involvement, and exhibit low drug immunogenicity after intravenous injection administration.

### DESCRIPTION OF DRAWINGS

FIGS. 1A-1D illustrate schematic maps of vectors pscAAV-CAR-Gluc, pscAAV-CAR-MutC-Gluc, pscAAV-CAR-MutA-Gluc, and pscAAV-CAR-MutG-Gluc, respectively.

FIG. 2 illustrates the changes in Gluc levels in BHK-21 cells transfected respectively with vectors, pscAAV-CAR-Gluc, pscAAV-CAR-MutC-Gluc, pscAAV-CAR-MutA-Gluc and pscAAV-CAR-MutG-Gluc, as determined in an in vitro cell culture assay and compared with BHK-21 cells that were not transfected with a plasmid (i.e., blank control). ** indicates p<0.01.

FIGS. 3A-3C illustrate the Gluc levels, as detected respectively in brain tissue (FIG. 3A), heart tissue (FIG. 3B), and liver tissue (FIG. 3C) dissected from mice, after IV injection of a recombinant AAV vector carrying CAR promoter or CAR-Mut promoter (SEQ ID NO: 1). ** indicates p<0.01.

FIGS. 4A-4D illustrate schematic maps of vectors, pRDAAV-CMV-EGFP, pRDAAV-CAR-Mut-EGFP, pRDAAV-CAR-Mut-coGAA, and pRDAAV-CAR-Mut-coGAA-2×142-3P, respectively.

FIG. 5 illustrates the levels of GAA enzyme activity in cells infected or uninfected with virus, as measured in an in vitro cell culture assay. BHK cells: blank BHK-21 cells uninfected with virus; rAAV9-CAR-Mut-coGAA-142-3p: BHK-21 cells infected with recombinant AAV9 virus, rAAV9-CAR-Mut-coGAA-2×142-3P; ** indicates p<0.01.

FIG. 6 illustrates the GAA enzyme activities in heart, liver, muscle, kidney, lung, and spleen tissues dissected from mice after a single IV injection administration of the recombinant AAV9 virus, rAAV9-CAR-Mut-coGAA-2×142-3P, as measured in an in vivo evaluation assay in GAA^{-/-} model mice. Model control group: model mice injected with PBS as a negative control; low-dose group: model mice injected with 5E12 vg/kg of the recombinant AAV9 virus; medium-dose group: model mice injected with 1.1E13 vg/kg of the recombinant AAV9 virus; high-dose group: model mice injected with 3E13 vg/kg of the recombinant AAV9 virus; wild-type control group: 129 wild-type mice.

FIGS. 7A-7D illustrate histopathological staining after a single IV injection administration of the recombinant AAV9 virus rAAV9-CAR-Mut-coGAA-2×142-3P, as assayed in Experiment 1 for in vivo evaluation of GAA^{-/-} model mice. FIG. 7A shows the results of H&E staining of liver tissue (top row: 100× magnification; bottom row: 400× magnification); FIG. 7B shows the results of H&E staining of cardiomyocytes; FIG. 7C shows the results of H&E staining of skeletal muscle cells; and FIG. 7D shows the PAS staining of skeletal muscle cells and cardiomyocytes. In the figures, Gaa^{-/-}: tissue sections of the model mice in the PBS administration group; LD: tissue sections of the model mice administered with a low dose of the recombinant AAV9 virus; MD: tissue sections of the model mice administered with a medium dose of the recombinant AAV9 virus; HD: tissue sections of the model mice administered with a high dose of the recombinant AAV9 virus; AAV treated: tissue sections of the model mice treated with the recombinant AAV9 virus.

FIGS. 8A-8C illustrate histopathological staining after a single IV injection administration of the recombinant AAV9 virus rAAV9-CAR-Mut-coGAA-2×142-3P, as assayed in Experiment 2 for in vivo evaluation of GAA^{-/-} model mice. FIG. 8A shows the results of PAS staining of brain tissue; FIG. 8B shows the results of PAS staining of spinal cord tissue; and FIG. 8C shows the results of PAS staining of cerebellar tissue. In the figures, Gaa^{-/-}: tissue sections of the model mice in the PBS administration group; WT: tissue sections of 129 wild-type mice; 3E13 vg/kg: tissue sections of the model mice injected with 3E13 vg/kg of the recombinant AAV9 virus; and 6.8E13 vg/kg: tissue sections of model mice injected with 6.8E13 vg/kg of the recombinant AAV9 virus.

FIG. 9 illustrates the levels of GAA enzyme activity in brain tissues after a single IV injection administration of the recombinant AAV9 virus rAAV9-CAR-Mut-coGAA-2×142-3P, as measured in Experiment 2 for in vivo evaluation of GAA^{-/-} model mouse. In the figure, model control: model mice in the PBS administration group; 6.8E13 vg/kg: model mice injected with 6.8E13 vg/kg of the recombinant AAV9 virus.

FIG. 10 illustrates survival curves of mice after a single IV injection of PBS or the recombinant AAV9 virus rAAV9-CAR-Mut-coGAA-2×142-3P, as plotted in Experiment 4 for in vivo evaluation of GAA^{-/-} model mouse. In the figure, Gaa^{-/-}: model mice in the PBS administration group; 1.1E13 vg/kg AAV treatment: model mice injected with 1.1E13 vg/kg of the recombinant AAV9 virus.

### DETAILED DESCRIPTION OF THE INVENTION

The present invention discloses a gene therapy construct, a pharmaceutical composition and a method for the treatment of a subject with Pompe disease or acid glucosidase deficiency. In particular, the present invention relates to the construction, preparation and use of a recombinant AAV vector for delivering GAA.

Unless otherwise defined hereinafter, all technical and scientific terms used in the specification have the same meaning as is commonly understood by those ordinarily skilled in the art to which the present invention pertains. All publications, patent applications, patents and other references referred to herein are incorporated by reference in their entirety. Furthermore, the materials, methods and examples described herein are illustrative only and are not intended to be limiting. Other features, objects and advantages of the present invention will be apparent from the specification and the accompanying drawings and from the appended claims.

### Definition

The term “about”, when used in conjunction with a numerical value, is intended to encompass numerical values within a range having a lower limit of 5% less and an upper limit of 5% greater than the specified numerical value. The term is also intended to encompass numerical values within a range of ±1%, ±0.5%, or ±0.1% of the specified numerical value.

As used herein, the terms “comprise” or “include” are intended to include the elements, integers or steps, but not to exclude any other elements, integers or steps.

As used herein, the expressions “a first”, “a second” or “a third” are used to distinguish the elements referred to and, unless otherwise indicated, are not indicative of the requirement that the elements referred to have particular numbers or in any particular order or position.

As used herein, the expression “and/or” is used to represent any one of the listed related items, or any and all possible combinations of the listed related items.

As used herein, a recombinant adeno-associated virus can be represented solely by the AAV virus serotype from which its capsid is derived, or by the AAV virus serotypes from which its capsid and genomic ITR sequence are derived. In the second scenario, the symbol “/” is utilized here for separation. It is placed before the serotype from which the capsid is derived and after the serotype from which the ITR is derived. Therefore, for instance, in the case of “a recombinant AAV9,” the number 9 signifies that the recombinant adeno-associated virus has a capsid from AAV9 serotype. Conversely, in the case of “a recombinant AAV2/9,” the number preceding the slash indicates the recombinant adeno-associated virus has a wild-type or variant ITR sequence from AAV2, while the number following the slash indicates that the recombinant adeno-associated virus has a capsid protein from AAV9.

The terms “acid alpha-glucosidase” or “acid glucosidase” or GAA are used interchangeably herein to refer to: a lysosomal enzyme capable of hydrolyzing the alpha-1-4 bond in maltose and other linear oligosaccharides, thereby degrading excess glycogen in lysosomes. When the gene encoding GAA is expressed in a cell, the GAA polypeptide will be synthesized in the cytoplasm and glycosylated in the ER, with a high-mannose-type sugar chain linked to N-terminus. In the Golgi apparatus, the high-mannose sugar chain on GAA can be further modified to add mannose-6-phosphate (M6P). Through the interaction of M6P with M6P receptor, GAA is delivered to lysosome, where it functions in glycogen degradation.

Examples of GAA include, but are not limited to, an enzyme protein having the amino acid sequence of a full-length wild-type (natural) human GAA (as represented by the Unipro database accession number UniProtKB-P10253), mature forms thereof, variants thereof (e.g., variants with conserved amino acid substitutions), and fragments thereof. Human GGA has a conserved 6-mer peptide of WIDMNE at amino acid residues 516-521, which is required for GAA protein activity. Variants and fragments of GAA may also be used herein as long as said variants or fragments retain glycogen-hydrolyzing activity and provide, for example, at least about 50%, at least about 60%, at least about 70%, at least about 75%, at least about 80%, at least about 90%, or about 100%, or greater than 100% of the enzyme activity level of a full-length wild-type (natural) human GAA.

In one embodiment of the present invention, a GAA polypeptide comprises an amino acid sequence of SEQ ID NO: 13, an amino acid sequence of residues 70-952 of SEQ ID NO: 13, an amino acid sequence of residues 123-952 of SEQ ID NO: 13, an amino acid sequence of residues 204-952 of SEQ ID NO: 13, or an amino acid sequence having at least 90%, or at least 95%, 96%, 97%, 98%, 99% or greater identity to any of the preceding sequences. The first 27 amino acids of a human GAA polypeptide is a typical signal peptide for lysosomal proteins and secretory proteins. The GAA can be targeted to the lysosomes via the signal peptide. Thus, in one embodiment, a GAA polypeptide of the present invention comprises a signal peptide that targets lysosomes, such as a natural signal peptide sequence from a human GGA polypeptide. In another embodiment, a GAA polypeptide of the present invention comprises a signal peptide from a heterologous protein targeting lysosomes.

In some embodiments of the present invention, a polynucleotide sequence encoding a GAA polypeptide comprises a wild-type GAA nucleic acid sequence. In a further embodiment of the present invention, a polynucleotide sequence encoding a GAA polypeptide is codon-optimized for human (i.e., codon-optimized for expression in human cells), for example, to enhance the in vivo expression and/or stability of said polynucleotide. Preferably, a polynucleotide sequence encoding GAA comprises the polynucleotide sequence of SEQ ID NO: 10.

The term “ETR” or “enzyme replacement therapy” means, as used herein, a therapeutic procedure for the treatment of Pompe disease or acid glucosidase deficiency in which a recombinant GAA protein is administered to a subject in need thereof. Recombinant GAA proteins for ETR can be produced in engineered mammalian cell lines, such as CHO cells, or in the milk of transgenic animals, such as transgenic rabbits.

As used herein, the term “conservative” alteration of an amino acid or a nucleotide refers to a neutral or near-neutral alteration of an amino acid or a nucleotide, which results in that the protein or nucleic acid molecule comprising the amino acid alteration or nucleotide alteration substantially retains original function thereof. For example, a conservative amino acid substitution is the replacement or substitution of an amino acid with a different amino acid with a side chain having similar biochemical properties (e.g., charge, hydrophobicity, and size). Such conservatively modified variants are additional to and not exclusive of polymorphic variants, interspecies homologs, and alleles. The following eight groups comprise amino acids with mutually conserved substitutions: 1) alanine (A), glycine (G); 2) aspartic acid (D), glutamic acid (E); 3) asparagine (N), glutamine (Q); 4) arginine (R), lysine (K); 5) isoleucine (I), leucine (L), methionine (M), valine (V); 6) phenylalanine (F), tyrosine (Y), tryptophan (W); 7) serine(S), threonine (T); and 8) cysteine (C), methionine (M) (cf. e.g., Creighton, Proteins (1984)). Those skilled in the art can readily detect the conservation of amino acid or nucleotide alterations in a particular polypeptide sequence or nucleotide sequence by conventional technical means, such as functional assay tests.

The term “functionally linked”, also referred to as “operably linked”, means that the designated components are in a relationship that allows them to function in their intended manner.

The term sequence “identity” is used to describe the structural similarity between two amino acid sequences or two polynucleotide sequences. To determine the percentage identity of two amino acid sequences or two nucleic acid sequences, the sequences can be aligned for optimal comparison purposes (e.g., a gap may be introduced in one or both of the first and second amino acid sequences or nucleic acid sequences for optimal alignment, or non-homologous sequences may be discarded for comparison purposes). In a preferred embodiment, the length of the reference sequence aligned for comparison purposes is at least 30%, preferably at least 40%, more preferably at least 50%, 60% and even more preferably at least 70%, 80%, 90%, 100% of the length of the reference sequence. The amino acid residues or nucleotides at the corresponding amino acid positions or nucleotide positions are subsequently compared. When a position in the first sequence is occupied by the same amino acid residue or nucleotide at the corresponding position in the second sequence, then said molecule is identical at this position.

Mathematical algorithms can be used to implement sequence comparisons between two sequences and to calculate percent identity. In a preferred embodiment, the Needlema and Wunsch ((1970) J. Mol. Biol. 48:444-453) algorithm that has been integrated into the GAP program of the GCG software package (available at http://www.gcg.com) is employed, using the Blossum 62 matrix or the PAM250 matrix with gap weights of 16, 14, 12, 10, 8, 6, or 4 and length weights of 1, 2, 3, 4, 5, or 6 to determine the percent identity between two amino acid sequences. In a further preferred embodiment, the percentage identity between two nucleotide sequences is determined employing the GAP program in the GCG software package (available at http://www.gcg.com), using the NWSgapdna.CMP matrix and gap weights of 40, 50, 60, 70 or 80 and length weights of 1, 2, 3, 4, 5 or 6. A particularly preferred set of parameters is the Blossum 62 scoring matrix with a gap penalty of 12, a gap extend penalty of 4 and a frameshift gap penalty of 5 (and unless otherwise stated, the set of parameters should be used).

The percent identity between two amino acid sequences or nucleotide sequences can also be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

The term “host cell” refers to a cell into which an exogenous polynucleotide has been introduced, including the progeny of such a cell. In some embodiments, the host cell is any type of cell system that can be used to produce a recombinant AAV vector of the present invention, e.g., mammalian cells (e.g., HEK 293 cells for production of a recombinant AAV via a triple-plasmid packaging system) and insect cells (e.g., sf9 cells for the production of a recombinant AAV via a baculovirus packaging system).

The term “regulatory sequence” or “expression control sequence” refers to a nucleic acid sequence that induces, represses or otherwise controls the transcription of a nucleic acid sequence encoding a protein, to which the nucleic acid sequence encoding the protein is operably linked. Regulatory sequences can be, for example, a start sequence, an enhancer sequence, an intron sequence and a promoter sequence.

The terms “exogenous” or “heterologous” are used interchangeably when describing nucleic acids or proteins, which means that the nucleic acids or proteins are not naturally occurring in the locations in which they are found in the chromosome or host cell. An exogenous nucleic acid sequence also refers to a sequence derived from and inserted into the same host cell or subject, but in an unnatural state, e.g., where the sequence is present at a different copy number or under the control of a different regulatory element.

As used herein, an “isolated” polynucleotide (e.g., isolated DNA or isolated RNA) means that the polynucleotide is isolated, at least in part, from at least some other components in a natural organism or virus comprising thereof. In some embodiments, the “isolated” nucleic acid is enriched at least about 10-fold, 100-fold, 1,000-fold, 10,000-fold, or more relative to the starting material.

As used herein, an “isolated” polypeptide is a polypeptide that is at least partially isolated from at least some other components in a natural organism or virus comprising thereof. In some embodiments, the “isolated” polypeptide is enriched at least about 10-fold, 100-fold, 1,000-fold, 10,000-fold, or more relative to the starting material.

As used herein, “isolating” or “purifying” a virus vector means that the virus vector is partially isolated from at least some components in the starting material comprising thereof. In some embodiments, the “isolated” virus vector is enriched at least about 10-fold, 100-fold, 1,000-fold, 10,000-fold, or more relative to the starting material.

As used herein, the term “virus vector” refers to a virus particle (e.g., an AAV virus particle) that is capable of serving as a vehicle for the delivery of a nucleic acid of interest. Typically, a virus vector comprises a capsid and a viral genome (e.g., viral DNA) packaged therein, and the nucleic acid of interest to be delivered is inserted in the viral genome. In the case of a recombinant AAV virus vector, in order to produce a recombinant virus particle that can deliver the nucleic acid of interest to a tissue or cell, it is usually necessary to retain only the inverted terminal repeat (ITR) as cis-element in the genome, while other sequences required for viral packaging can be provided in trans. Accordingly, in some embodiments, the recombinant AAV virus vectors of the present invention comprise a capsid and a recombinant viral genome packaged therein, wherein the recombinant viral genome comprises or consists of one or more exogenous nucleotide sequences located between two AAV ITR sequences. The two ITR sequences located at the 5′ and 3′ ends of the recombinant viral genome (i.e., the 5′ ITR and the 3′ ITR) may be the same or different.

The term AAV “inverted terminal repeat” (ITR) means, as used herein, a cis-acting element from an AAV virus genome that plays an important role in the integration, rescue, replication, and genome packaging of AAV viruses. The ITR sequence of a natural AAV virus comprises a Rep protein binding site (RBS) and a terminal resolution site (trs), which can be recognized and bound by the Rep protein, and create a cut at trs. The ITR sequence also can forms a unique “T” letter-type secondary structure, which plays an important role in the life cycle of AAV viruses. The earliest isolated AAV virus, AAV2, has a 145-bp “inverted terminal repeat” (ITR) with a palindromic-hairpin structure located at both ends of the genome. Subsequently, different ITR sequences have been found in various AAV virus serotypes, but all of them form a hairpin structure and have a Rep binding site. Conventional recombinant AAV virus vectors based on these wild-type ITR sequences are generally single-stranded AAV vectors (ssAAV), with the viral genome packaged in a single-stranded form in an AAV capsid. Unlike such ssAAVs, it has been found that by modifying ITRs by deleting trs sequence and optionally D sequence from an ITR sequence on one side of the AAV virus, the genome carried by the packaged recombinant AAV virus vector can be self-complementary to form a double-stranded (Wang Z et al., Gene Ther. 2003; 10(26):2105-2111; McCarty DM et al., Gene Ther. 2003; 10(26):2112-2118). The virus thus packaged is a double-stranded AAV virus, i.e., scAAV (self-complementary AAV) virus. scAAV virus vector has a smaller packaging capacity, which is about 2.2 kb-2.5 kb, and only half of that of ssAAV virus vector, but has a higher transduction efficiency after infecting cells.

As used herein, the term ITR in relation to AAV covers both wild-type ITR and variant ITR. wild-type ITR can be derived from any natural AAV virus, such as AAV2 virus. Wild-type ITR comprises a Rep protein binding site (RBS) and a terminal resolution site (trs), which can be recognized and bound by the Rep protein, and generate a cut at the trs. The wild-type ITR sequence can form a unique “T” letter-type secondary structure, and plays an important role in the life cycle of AAV virus. As used herein, a variant ITR is a non-natural ITR sequence that may, for example, be derived from any wild-type AAV ITR sequence and comprise one or more nucleotide deletions, substitutions, and/or additions, and/or truncations, relative to the wild-type ITR, but is still functional, i.e., is capable of being used to generate an ssAAV virus vector or an scAAV virus vector. In some embodiments, a variant ITR is an AAV ITR sequence (also referred to herein as ΔITR) that is deleted a functional trs site and optionally a D region sequence. In some embodiments, wild-type ITR is used in combination with ΔITR to generate a self-complementary recombinant AAV virus vector (scAAV). In other embodiments, a combination of two wild-type ITRs is used to generate a single-stranded recombinant AAV virus vector (ssAAV).

The AAV proteins VP1, VP2 and VP3 are capsid proteins that interact to form the AAV capsid. AAV viruses of different serotypes have different tissue tropism of infection and can be used to transport an exogenous gene to specific organs and tissues by selecting the source serotype from which the capsid of the recombinant AAV virus vector derived (Wu Z et al., Mol Ther. 2006; 14(3): 316-327). In the present invention, the recombinant AAV virus vector can have different targeting properties by selecting the source serotype of the capsid. In some embodiments, the capsid of the recombinant AAV virus is derived from an AAV serotype that has a targeting property to neuronal cells. In one embodiment, the recombinant AAV virus vector comprises a capsid from AAV9. In a further embodiment, the recombinant AAV virus vector comprises a capsid from AAV9 and ITRs from AAV2.

The term “immune-related miRNA” is an miRNA that is preferentially expressed in cells of the immune system, e.g., antigen-presenting cells. In some embodiments, the expression level of the immune-related miRNA in immune cells is higher than that in non-immune cells (e.g., a reference cell, e.g., HEK293 cells), in particular at least 2-fold, 3-fold, 4-fold, 5-fold, 6-fold, 7-fold, 8-fold, 9-fold, or 10-fold higher. In some embodiments, the immune system cells expressing the immune-related miRNA are B cells, T cells, T killer cells, T helper cells, dendritic cells, macrophages, monocytes, vascular endothelial cells, or other immune cells. In some embodiments, the immune-related miRNA is miR-142-3P. miR-142-3p is an miRNA that is highly expressed in cells derived from hematopoietic stem cell lines. Immune cells are all differentiated from hematopoietic stem cell lineage, so using the principle of miRNA on the inhibition of gene expression, the expression of a gene carrying the miR-142-3p target sequence is significantly inhibited in immune cells, thereby the probability that the organism will generate an immune response against the gene expression product is reduced.

The term “treatment” refers to clinical interventions intended to modify the natural course of disease in the individual being treated. Desired therapeutic effects include, but are not limited to, preventing the emergence or recurrence of the disease, alleviating symptoms, minimizing any direct or indirect pathological consequences of the disease, preventing metastasis, decreasing the progression rate of the disease, ameliorating or moderating the state of the disease, and alleviating or improving prognosis. In some embodiments, the recombinant AAV virus of the present invention after administration, preferably after systemic administration to a subject with Pompe disease or a GAA deficiency, reduces lysosomal glycogen storage in multiple involved tissues (in particular, skeletal muscle, cardiac muscle, diaphragm, and central nervous system) of the subject. In some embodiments, the recombinant AAV virus of the present invention after administration, preferably after systemic administration to a subject with Pompe disease or a GAA deficiency, ameliorates central nervous system damage in the subject. In some embodiments, the recombinant AAV virus of the present invention after administration, preferably after systemic administration to a subject with Pompe disease or a GAA deficiency, ameliorates skeletal muscle damage, and myocardial muscle damage in the subject. In some embodiments, the recombinant AAV virus of the present invention after administration, preferably after systemic administration to a subject with Pompe disease or a GAA deficiency, ameliorates pathological changes due to the disease in the subject's nervous system (including cerebral, spinal cord and/or cerebellar tissues). In some embodiments, glycogen accumulation in glial cells in brain tissue is improved. In another embodiment, the recombinant AAV virus of the present invention after administration, preferably after systemic administration to a subject with Pompe disease or a GAA deficiency, prolongs the survival of the subject.

As used herein, “prevention” includes the inhibition of the onset or progression of a disease or a particular symptom of a disease. In some embodiments, a subject with a predisposition to Pompe disease is a candidate for a prophylactic regimen. Typically, the term “prevention” refers to a hospital intervention that is implemented prior to the onset of at least one symptom of the disease. Thus, in one embodiment, prevention comprises administration of the gene therapy drug of the present invention to a subject with a defective GAA gene prior to the onset of the symptoms of Pompe disease in order to slow the progression of the disease or to prevent the disease from appearing.

Various aspects of the present invention are described below.

### I. Constructs for Gene Therapy

### Constitutive CAR-Mut Promoter

A promoter is a specific DNA sequence that is recognized, bound by RNA polymerase, and initiates transcription. Eukaryotic class II promoters are involved in the transcriptional control of protein-coding genes, usually located at upstream of the coding regions of the genes, and regulate the timing and location of gene transcription through interactions with transcription factors (TFs). This type of promoter comprises five types of elements: basic promoter, initiator, upstream element, downstream element and response element. Different combinations and sequence changes of these elements confer multiple effects on the functional activities of promoters (Fang TANG, Huizhen TU, Research progress on eukaryotic promoters [J]. Forestry Science and Technology Development. 2015, 29(2):7-12).

In one aspect of the present invention, there is provided a synthetic mutant constitutive promoter, CAR-Mut. The constitutive promoter of the present invention can efficiently initiate the expression of an exogenous gene in a wide variety of tissues, and is thus particularly suitable for application in the therapeutic methods of the present invention to address the peripheral organ involvement of Pompe disease and to reduce the burden in the central nervous system.

In one embodiment, the present invention provides a mutant promoter comprising a polynucleotide selected from the group consisting of:
(i) a polynucleotide of SEQ ID NO: 4.
(ii) a polynucleotide having at least 95%, 96%, 97%, 98%, 99%, or 99.5% identity to SEQ ID NO: 4.
(iii) a polynucleotide obtained by substitution, deletion or addition of one or several nucleotides to the polynucleotide of SEQ ID NO: 4,
and wherein the polynucleotide has a mutation at positions 562-572 of SEQ ID NO:4 or at positions corresponding thereto, preferably said mutation is a mutation from T to C or G or A at position 568 or at a corresponding position, and more preferably a T to C mutation.

In a preferred embodiment, the mutant promoter of the present invention, compared with a reference promoter consisting of a corresponding polynucleotide without the mutation, increases the expression of a gene of interest to which it is functionally linked, e.g., increases the expression of the gene of interest by 1%-70%, e.g., by at least 5%, 10%, 20%, 30%, 40%, or at least 50%, 60%.

In a further preferred embodiment, the mutant promoter of the present invention increases the expression of the gene of interest to which it is functionally linked in mammalian cells or tissues relative to a reference promoter, e.g., increases the expression of the gene of interest in mammalian peripheral tissues and/or central nervous tissues, in particular, increases the expression in mammalian tissues selected from the heart, the liver and/or the brain. Preferably the mammal is a human or a non-human mammal, e.g., mice, rats and non-human primates.

In further embodiments, the promoter comprises a nucleotide sequence selected from any one of SEQ ID NOs: 1 to 3, or a nucleotide sequence differing therefrom by one or several nucleotide substitutions, deletions and/or additions and having equivalent promoter activity. Preferably, the promoter comprises or consists of the nucleotide sequence of SEQ ID NO: 1.

A person skilled in the art can use any promoter functionality assays known in the art (e.g., the luciferase reporter gene expression assay of Example 1) to determine whether any two promoters have equivalent promoter activity. In one embodiment, as compared to a reference promoter (e.g., SEQ ID NOs: 1-3), a promoter to be tested can be considered to have equivalent promoter activity if the promoter to be tested has the same or substantially the same activity under the same assay conditions, e.g., ±10% of the reference promoter activity, preferably ±5% of the reference promoter activity, or more preferably ±1% of the reference promoter activity.

In some aspects, the present invention also encompasses expression cassettes, recombinant vectors and host cells comprising the promoter and a coding nucleotide sequence functionally linked thereto, and compositions and methods for delivering a coding polynucleotide to a mammalian cell or individual using the expression cassettes, vectors or host cells.

### Expression Construct

In one aspect, the present invention provides an expression construct. The expression construct of the present invention comprises a promoter of the present invention and can be advantageously used for the expression of GAA-coding nucleic acid sequence in desired tissues or cells of patients with Pompe disease or acid glucosidase deficiency.

In one embodiment, the expression construct of the present invention comprises the following elements functionally linked to each other in a transcriptional direction:
any CAR-Mut promoter of the present invention, in particular the promoter of SEQ ID NO: 1,
optionally, a Kozak sequence,
a polynucleotide encoding a gene of interest, e.g., a polynucleotide sequence encoding acid alpha-glucosidase (GAA), preferably a human codon-optimized sequence coding for a human GAA polypeptide, more preferably the sequence of SEQ ID NO:10,
optionally, at least one (e.g., 2 to 4) immune-related miRNA binding site, in particular a miR-142 binding site, e.g., a miR-142 binding site comprising at least one (e.g., one or two) sequence of SEQ ID NO:11,
optionally, a transcription terminator, e.g., a poly A signaling sequence, preferably selected from the group consisting of a SV40 late polyA sequence, a rabbit B-globin polyA sequence, a bovine growth hormone polyA sequence, or any variant thereof, and more preferably a bovine growth hormone polyA sequence comprising SEQ ID NO: 13 or a sequence having at least 95% identity thereto.

In some embodiments, the expression construct further comprises two ITR sequences. For example, from the 5′ end to the 3′ end, the expression construct may comprise elements arranged as follows: 5′ITR-promoter-GAA coding sequence-miRNA binding site-polyA-3′ITR. In some embodiments, the 5′ITR and 3′ITR are identical. In another embodiment, the 5′ITR and the 3′ITR are different and one of them (preferably the 3′ITR) is a AITR that lacks a functional trs site. In one embodiment, the 5′ITR and the 3′ITR in the expression construct are identical and both comprise or consist of a sequence of SEQ ID NO: 5. In a further embodiment, the 5′ITR and the 3′ITR in the expression construct are different, wherein the 5′ITR comprises or consists of the sequence of SEQ ID NO: 5 and the 3′ITR comprises or consists of the sequence of SEQ ID NO: 6.

The promoter used in the expression construct of the present invention may be a CAR-Mut promoter as described in any of the above embodiments of the present invention. In a preferred embodiment, the promoter comprises or consists of the nucleotide sequence of SEQ ID No:1. In another preferred embodiment, the promoter comprises or consists of the nucleotide sequence of SEQ ID NO: 2. In another preferred embodiment, the promoter comprises or consists of the nucleotide sequence of SEQ ID NO:3.

The expression construct of the present invention can in one embodiment comprise a Kozak sequence located upstream of a start codon of a nucleic acid sequence encoding GAA to facilitate the translation of GAA. A Kozak sequence for use in the present invention may be a consensus sequence defined as GCCRCC, wherein R is a purine (i.e. A or G) and wherein the sequence is located upstream of a start codon. In a preferred embodiment, in the nucleic acid sequence of the expression construct of the invention, the Kozak sequence has a 5′-GCCACC-3′ sequence. Other different Kozak sequences may also be used. Kozak sequences can be screened from sequence libraries, and the enhancement of translation efficiency can be assessed using conventional means known in the art. For example, a recombinant nucleic acid comprising a reporter gene or a recombinant GAA gene and a different Kozak sequence can be constructed and introduced into a host cell, such as a BHK cell. After a period of time, the expression level of the reporter gene or the level of GAA enzyme activity can be examined in the cells or in the culture supernatant, and compared with that of the recombinant nucleic acid having a reference Kozak sequence to determine the translation enhancement efficiency of the tested Kozak sequence.

In some embodiments, the expression construct of the present invention further comprises one or more immune-related miRNA binding sites, i.e., miRNA target sequences, located in the 3′UTR of the nucleic acid sequence encoding the desired GAA. Without being bound by any specific theory, the inclusion of the miRNA binding site in the expression construct allows for modulation (e.g., inhibition) of the expression of the desired gene in cells and tissues that produce the corresponding miRNA. Thus, in one embodiment, the expression construct of the present invention comprises one or more miRNA binding sites such that GAA expression can be down-regulated in a cell type-specific manner. In one embodiment, the expression construct of the present invention comprises one or more miRNA binding sites, and the miRNA is expressed in an antigen presenting cell, thereby reducing the GAA expression efficiency of the expression construct of the invention in the antigen presenting cell. In some embodiments, the one or more miRNA binding sites are located in the 3′ untranslated region (3′UTR) of the gene encoding GAA, e.g., located between the last codon of the nucleotide sequence encoding GAA and a poly A sequence.

In some embodiments, the expression construct comprises one or more (e.g., 1, 2, 3, 4, 5 or more) miRNA binding sites, and the miRNA binding site(s) down-regulates the expression of GAA gene in an immune cell (e.g., an antigen-presenting cell APC, such as a macrophage and a dendritic cell, etc.). Without being bound by specific theory, incorporation of such immune-related miRNA binding sites into the expression construct result in a reduced expression of the GAA gene of interest in antigen-presenting cells having the miRNA, thereby reducing or inhibiting the development of an anti-GAA immune response in a subject.

In some preferred embodiments, the expression construct comprises one or more miR-142 binding sites (also referred to herein as miR-142 target sequences), e.g., the miR-142-3P target sequence shown in SEQ ID NO: 11, or tandem repeat sequences thereof, e.g., 2, 3, 4, 5, 6 tandem repeats, preferably 2 tandem repeats, such as a miR-142-3P target sequence shown in SEQ ID NO: 12. In some embodiments, the miRNA binding site reduces expression of the recombinant AAV vector in antigen-presenting cells. In some embodiments, the miRNA binding site reduces the immunogenicity of a recombinant AAV vector. In some embodiments, a recombinant AAV vector comprising the miRNA binding site induces a low immune response in a subject. In other some embodiments, after administration, a recombinant AAV vector containing the miRNA binding site induces a lower anti-GAA serum titer in a subject compared to a control recombinant AAV vector lacking the miRNA binding site. Preferably, the administration is an intravenous administration. In one embodiment, the anti-GAA serum titer is determined 1-6 weeks, e.g., 5 weeks, after the administration, and preferably, the serum titer is reduced by about 1 to about 10-fold, e.g., about 2-fold, 3-fold, 4-fold, 5-fold, 6-fold, 7-fold, or 8-fold, relative to the control.

In some embodiments, the expression construct of the present invention comprises at least one poly A tail located downstream of the polynucleotide encoding a GAA and miRNA binding site. Any suitable poly A sequence can be used, including, but not limited to, hGHpolyA, BGHpolyA, SV40 late poly A sequence, rabbit B-globin polyA sequence, or any variant thereof. In a preferred embodiment, the poly A is BGHpolyA, for example, a polyA shown in SEQ ID NO: 7, or a polyA polynucleotide sequence having at least 80%, 85%, 90%, 95%, 96%, 97%, 98% or 99% nucleotide sequence identity to SEQ ID NO: 7.

The GAA-coding nucleic acid comprised in the expression construct of the present invention may be any polynucleotide encoding a functional GAA enzyme activity. In one embodiment, the nucleic acid encodes a human full-length GAA sequence, for example, the sequence of SEQ ID NO: 13, or a fragment thereof, for example, a GAA enzyme fragment that begins at any residue selected from residues 1-204 and terminates at residue 952 of SEQ ID NO: 14 or a GAA enzyme fragment having corresponding positions. Preferably, the GAA comprises a natural signal peptide that targets the lysosome (i.e., in the case of SEQ ID NO: 13, the signal peptide at amino acid positions 1-27). Alternatively, the GAA may comprise a heterologous signal peptide, e.g., a signal peptide derived from a human lysosome-targeting protein or a secreted protein. Examples of heterologous signal peptides that can be used in the present invention include, but are not limited to: signal peptides from immunoglobulins (e.g., IgG), cytokines (e.g., IL-2), insulin. See, for example, WO2018046774.

In some embodiments, an expression construct of the present invention comprises a nucleic acid sequence encoding GAA, wherein the nucleic acid sequence encodes a polypeptide having GAA enzyme activity, wherein the polypeptide comprises an amino acid sequence having at least 95%, at least 97%, at least 98%, or at least 99% or higher sequence identity to the sequence of SEQ ID NO: 13, or to the sequence at the amino acid positions 70-952 of SEQ ID NO: 13, or to the sequence at the amino acid positions 123-952 of SEQ ID NO: 13, or to the sequence at the amino acid positions 204-952 of SEQ ID NO: 13. Preferably, the polypeptide has about the same glycogen hydrolyzing activity as compared to the reference GAA protein shown in SEQ ID NO: 13, e.g., the GAA enzyme activity of the polypeptide is at least about 95%, at least about 96%, at least about 97%, 98%, 99%, or more of the enzyme activity of the reference GAA protein. Assays for determining GAA enzyme activity are known in the art. Those skilled in the art can employ any such assay to determine suitable GAA polypeptide that can be used in the expression construct, recombinant AAV virus vector, and method and use of the invention.

To facilitate the expression in human cells, the nucleic acid used to encode a GAA polypeptide is preferably codon-optimized. In one embodiment, the GAA-coding nucleic acid in the expression construct of the present invention comprises a polynucleotide sequence of SEQ ID NO: 13, or a polynucleotide sequence having at least about 95%, about 96%, about 97%, 98%, 99%, or higher nucleotide sequence identity to SEQ ID NO: 13.

In some aspects, the present invention also provides a vector comprising the expression construct of the present invention. In some embodiments, the vector is a plasmid (e.g., a plasmid for the production of recombinant virus particles). In some other embodiments, the vector is a virus vector, e.g., a recombinant AAV vector or a baculovirus vector. In some embodiments, the genome of the recombinant AAV vector is single-stranded (e.g., single-stranded DNA). In some embodiments, the genome of the recombinant AAV vector is self-complementary. In yet further embodiments, the vector is a baculovirus vector (e.g., Autographa californica nuclear polyhedrosis virus (AcNPV) vector).

In a further aspect, the present invention also provides host cells, such as mammalian cells or insect cells, comprising the expression construct or vector of the present invention. In some embodiments, the cells are used to produce recombinant AAV viruses.

### Recombinant AAV Vector

In one aspect, the present invention provides a recombinant AAV vector. The recombinant AAV vector of the present invention may be used, in particular, for the treatment of Pompe disease or acid glucosidase deficiency. In one embodiment, the recombinant AAV vector comprises a capsid and a nucleic acid inside the capsid. The nucleic acid also referred to herein as “the genome of the recombinant AAV vector”. The genome of the recombinant AAV vector comprises a plurality of elements including, but not limited to, two inverted terminal repeats (ITRs, i.e., 5′-ITR and 3′-ITR), and other elements located between the two ITRs, including a promoter, a heterologous gene, and a polyA tail. Preferably, at least one immune-related miRNA binding site may also be comprised between the two ITRs.

As used herein, adeno-associated virus (AAV) includes, but is not limited to, AAV of any serotype, such as AAV serotype types 1, 2, 3, 4, 5, 6, 7, 8, 9, 10, 11, and AAV having an artificially modified capsid protein. The genome sequences of the various serotypes and of the artificial AAVs, as well as the natural inverted terminal repeat (ITR) sequences thereof, the Rep proteins, and the capsid (also abbreviated as “cap”) proteins, are well known in the art. These sequences can be found in public databases such as GenBank or in the literatures.

In some embodiments, the present invention provides a recombinant AAV virus vector comprising a capsid, wherein the capsid consists of capsid proteins capable of crossing the blood-brain barrier, such as the capsid proteins from AAV9, AAVPHP.B, or AAVPHP.eB. In some embodiments, the recombinant AAV vector of the present invention transduces neuronal cells in the central nervous system (CNS) and also peripheral non-neuronal cells. In further embodiments, following systemic administration, the recombinant AAV vector targets and transduces muscle cells and neuronal cells. In yet further embodiments, following systemic administration, the recombinant AAV vector targets and transduces peripheral organs and central nervous system in a subject. In yet further embodiments, following systemic administration, the recombinant AAV vector targets and transduces a multiple of tissues in a subject (such as the brain, spinal cord, skeletal muscle, heart, and liver), and preferably, leads to increased expression of the exogenous gene of the interest (in this case, GAA) and/or higher enzyme activity in the targeted and transduced tissues, when compared to a control subject that has not received the recombinant AAV vector.

In some embodiments, the recombinant AAV vector in this invention features a capsid derived from the AAV9 serotype (referred to herein as AAV9 vector); and preferably, the recombinant AAV vector contains a wild-type or variant ITR sequence from AAV2 in its genome (referred to herein as AAV2/9 vector).

In some embodiments, the ITR sequences of the recombinant AAV vector of the present invention are both a full-length ITR (e.g., having a length of about 125-145 bp and comprising a functional Rep binding site (RBS) and a terminal resolution site (trs)). In some embodiments, the full-length functional ITRs are used to produce a single-stranded recombinant AAV vector (ssAAV). In some other embodiments, one of the ITRs of the recombinant AAV vector is truncated. In some embodiments, the truncated ITR lacks a functional terminal resolution site (trs) and is used to produce a self-complementary recombinant AAV vector (scAAV vector).

In some embodiments, the recombinant AAV vector of the present invention comprises a wild-type AAV ITR, for example, a wild-type AAV2 ITR, e.g., the ITR sequence shown in SEQ ID NO: 5. In some other embodiments, the recombinant AAV vector of the present invention comprises a variant ITR with one or more modifications, e.g., nucleotide additions, deletions, and/or substitutions, relative to a wild-type AAV ITR. For example, the variant ITR is an AITR that is truncated relative to a wild-type AAV2 ITR and lacks a functional trs site, e.g., AITR sequence shown in SEQ ID NO: 6.

Accordingly, in one aspect, the present invention provides a recombinant adeno-associated virus (AAV) vector, wherein the recombinant AAV vector comprises in its genome:
a. 5′ and 3′ AAV inverted terminal repeat (ITR) sequences, and
b. an expression construct located between the 5′ITR and the 3′ ITR, wherein the expression construct comprises the following elements functionally linked to each other in a transcriptional direction:
   any CAR-Mut promoter according to the present invention, in particular the promoter of SEQ ID NO: 1,
   optionally, a Kozak sequence,
   a polynucleotide encoding a human acid alpha-glucosidase (GAA),
   optionally, at least one (e.g., 2 to 8) immune-related miRNA binding sites, in particular a miR-142 binding site, for example, a miR-142 binding site comprising at least one (e.g., 1 or 2) copies of the sequence of SEQ ID NO:11,
a transcription terminator, e.g., a polyA signaling sequence, preferably selected from the group consisting of a SV40 late polyA sequence, a rabbit β-globin polyA sequence, a bovine growth hormone poly A sequence, or any variant thereof.

In some embodiments, the polynucleotide encoding GAA in the recombinant AAV vector is codon-optimized for human, preferably to enhance the in vivo expression efficiency and/or stability of the polynucleotide, and more preferably, the polynucleotide comprises the sequence of SEQ ID NO:10.

In some embodiments, the ITRs of the recombinant AAV virus vector are both a wild-type AAV2 ITR sequence, or one of the ITRs is an AA2 AITR sequence lacking a functional terminal resolution site (trs).

In some embodiments, the recombinant AAV vector is an ssAAV vector. In some other embodiments, the recombinant AAV vector is an scAAV vector.

In some embodiments, the recombinant AAV vector comprises a capsid protein from AAV9 serotype, preferably, the recombinant AAV vector is an AAV2/9 vector.

### II. Preparation of Recombinant AAV Vector

The packaging systems for AAV vectors are relatively mature in the art. This facilitates large-scale production of AAV vectors.

Currently commonly used packaging systems for AAV vectors mainly include a triple-plasmid co-transfection system, a system using an adenovirus as a helper virus, a packaging system using a Herpes simplex virus type 1 (HSV1) as a helper virus, and a baculovirus-based packaging system. Each of these packaging systems has its own characteristics, and a person skilled in the art can make a choice as appropriate.

The packaging system using triple-plasmid transfection is of high safety since there is no need to use a helper viruse. The system is the most widely used packaging system for AAV vectors, and is also the mainstream pipeline currently for production in the world. A slight disadvantage in the system is absence of an efficient large-scale transfection method. This limits the use of the triple-plasmid transfection system in the large-scale preparation of AAV vectors.

Yuan et al. established a large-scale packaging system for AAV using adenovirus as a helper virus (Yuan Z et al., Hum Gene Ther. 2011; 22(5):613-624). This system demonstrates high production efficiency. Nevertheless, the adenovirus utilized in the packaging system remains in trace amounts in the final AAV product, thereby impacting the safety of the finished AAV product.

The packaging system utilizing HSVI as a helper virus is another relatively commonly used system for AAV vector packaging. Zhijian WU and Conway et al. almost simultaneously proposed a strategy for packaging AAV2 vectors with HSVI as a helper virus (Zhijian W U, Xiaobing W U, et. al., Science Bulletin, 1999, 44 (5): 506-509; Conway J E et al., Gene Ther. 1999, 6:986-993). Subsequently, Wustner et al. introduced a strategy for packaging AAV5 vectors with HSV1 as a helper (Wustner J T et al., Mol Ther. 2002, 6 (4): 510-518). Building on this, Booth et al. employed two HSV1 viruses to respectively carry the AAV rep/cap gene(s) and the AAV inverted terminal repeat (ITR)/exogenous gene expression cassette, and then co-infected production cells with the recombinant HSV1 viruses to package and produce AAV viruses (Booth M J, et al., Gene Ther. 2004; 11:829-837). Thomas et al. further developed a suspension cell system for dual HSV1-based AAV production (Thomas D L et al., Gene Ther. 2009; 20:861-870), which allows for the production of AAV viruses on a much larger scale.

Urabe et al. created a baculovirus-based AAV packaging system, which utilizes three different baculoviruses to carry AAV structural genes, AAV nonstructural genes, and AAV ITR/exogenous gene(s) cassette, respectively. Subsequently, considering the instability of baculoviruses carrying foreign genes, the number of baculovirus types required in the AAV production system has been progressively reduced, from initially three types of baculoviruses to two types or one type of baculoviruses (Chen H., Mol Ther. 2008, 16 (5): 924-930; Galibert L. et al., J Invertebr Pathol. 2011; 107 Suppl: S80-93), and to one kind of baculoviruses in combination with an inducible cell line (Mietzsch M et al., Hum Gene Ther. 2014; 25:212-222, Mietzsch M et al., Hum Gene Ther. 2015; 26(10):688-697).

The recombinant AAV virus vector of the present invention can be produced using any suitable method known in the art. In one embodiment, the recombinant AAV virus of the present invention is produced using a triple-plasmid packaging system. In another embodiment, the recombinant AAV virus of the present invention is produced using a baculovirus packaging system.

Accordingly, in one aspect, the present invention provides a cell comprising (i) a first vector encoding one or more adeno-associated virus rep proteins and/or one or more adeno-associated virus cap proteins; and (ii) a second vector comprising any of the expression constructs of the present invention described herein. The cells of the present invention may be used in the production of recombinant AAV virus vector of the present invention.

In another aspect, the present invention also provides a method of producing a recombinant AAV virus vector, wherein the method comprises the steps:
(i) providing a cell comprising (i) a first vector encoding one or more adeno-associated virus rep proteins and/or one or more adeno-associated virus cap proteins; and (ii) a second vector comprising any of the expression constructs of the present invention;
(ii) culturing the cell under conditions that permit packaging of a recombinant AAV; and
(iii) harvesting the cultured host cell or the culture medium to collect the recombinant AAV virus vector.

In one embodiment of the cells and production methods described above, the first vector is a plasmid, and the second vector is a plasmid; and the cell is a mammalian cell, optionally wherein the mammalian cell is a HEK293 cell. Where appropriate, the cell may provide other functions or partial functions required for the production of infectious recombinant AAV virus particles. In cases where the cell provides only partial functions, in some embodiments, the cell further comprises a third helper plasmid vector. The cell of the present invention can be readily prepared by transient co-transfection of the first plasmid vector, the second plasmid vector, and/or the third helper plasmid. In some embodiments where the functions required for the production of infectious AAV particles are provided by adenoviral genes, the third helper plasmid provides adenoviral genes VA, E2A, and E4; and the remaining adenoviral gene products required for the production are provided by host cells stably expressing adenoviral E1 gene. See, e.g., T Matsushita et al, Adeno-associated virus vectors can be efficiently produced without helper virus. Gene Therapy (1998) 5, 938-945.

In another embodiment of the cells and production methods described above, the first vector is a baculovirus vector and the second vector is a baculovirus vector; and the cell is an insect cell, optionally wherein the insect cell is a sf9 cell. In some embodiments, the AAV Rep proteins and the AAV Cap proteins are provided by two separate types of the first baculovirus vector; and in some other embodiments, the AAV Rep proteins and the AAV Cap proteins are provided at the same time by a single type of the first baculovirus vector. In some embodiments, the cells of the present invention may be created by producing two separate types of baculoviruses that each encode the GAA gene of interest and the AAV Rep and Cap proteins, and then co-infecting Spodoptera frugiperda (Sf9) insect cells with these two types of baculoviruses using e.g., a Bac-to-AAV system. See, e.g., Galibert L. et al. J Invertebr Pathol. 2011; 107 Suppl: S80-93.

### III. Pharmaceutical Compositions

In a further aspect, the present invention provides a pharmaceutical composition comprising the recombinant AAV virus vector of the present invention. The pharmaceutical composition of the present invention preferably comprises a pharmaceutically acceptable excipient, diluent or carrier. The pharmaceutical composition of the present invention may be formulated in any suitable formulation form.

Examples of suitable pharmaceutically acceptable excipients, diluents, or carriers for formulation are well known in the art and include, for instance, phosphate buffered saline, water, emulsions such as oil/water emulsions, various types of wetting agents, sterile solutions, and the like. The formulations can be prepared using conventional methods and administered to a subject in an appropriate dosage. Once properly formulated, the composition can be delivered via routes such as intravenously, intraperitoneally, subcutaneously, intramuscularly, locally or intradermally. The specific route of administration is determined based on in particular the type of carrier present in the pharmaceutical composition. The dosage regimen is decided by the attending physician, taking into account various clinical factors. As is well known in the medical field, the dosage for any particular patient is influenced by many factors, including the body size, body surface area, age, gender of the patient, and the specific agent being administered, the timing and route of administration, the drug and the usage stage thereof, the presence of an infection or disease, general health status, and the combination of other drugs.

In some embodiments, the pharmaceutical composition of the present invention comprises a second agent. In some embodiments, the second agent is a recombinant GAA protein for ERT, such as a recombinant GAA protein produced from the milk of a transgenic animal or from a productive mammalian cell line. In some embodiments, the second agent is a bronchodilator.

In some other embodiments, the pharmaceutical composition of the present invention comprises a component capable of reducing a side effect (e.g., an antidrug immune response) upon administration of the drug. In some instances, the component is an immunosuppressant.

The pharmaceutical composition of the present invention can be administered by any suitable route, including systemic administration and topical administration. In a preferred embodiment, the pharmaceutical compositions of the present invention are administered systemically, in particular via intravenous injection. Accordingly, in one embodiment, the present invention provides a pharmaceutical composition comprising a recombinant AAV vector of the present invention, wherein the pharmaceutical composition is an intravenous-injection, or a lyophilized stable formulation suitable for preparing an intravenous injection. In some other embodiments, the pharmaceutical composition of the present invention is for local administration, such as directly administered to or near an organ or tissue to be treated in a subject.

### IV. Method for Treatment

In another aspect, the present invention relates to a method for treating a disease using a recombinant AAV vector of the present invention or a pharmaceutical composition comprising the recombinant AAV vector. In one embodiment, the disease is Pompe disease. In another embodiment, the disease is acid alpha glucosidase deficiency. In one embodiment, the method comprises: administering to a subject in need thereof any of the recombinant AAV vectors of the present invention or any of the pharmaceutical compositions of the present invention. The recombinant AAV vector or the pharmaceutical composition can be administered by any suitable route, including, but not intramuscular, limited to, subcutaneous, intraspinal, intracerebroventricular, intrathecal, intravenous, intradiaphragmatic, intrathoracic, intraperitoneal routes. Preferably, the recombinant AAV vector or the pharmaceutical composition of the present invention is delivered to the subject by systemic administration, particularly via intravenous administration. In some embodiments, the treatment is therapeutic. In some other embodiments, the treatment is prophylactic. In some embodiments, the subject is a mammal, wherein the mammal is particularly a human, a primate, a dog, a horse, a cow, and especially a human subject.

In some embodiments where the method involves treatment of a subject having Pompe disease, the treatment comprises one or more of (1) preventing or delaying the onset of Pompe disease; (2) reducing the severity of Pompe disease; (3) reducing or preventing the onset and/or worsening of at least one of the symptoms of Pompe disease; (4) ameliorating Pompe disease-associated neurodegeneration and/or subject behavior; and (5) prolonging the survival of the subject. Subjects having Pompe disease who can be treated include patients with IOPD and LOPD. In some embodiments, the subject is an IOPD patient. In yet further embodiments, the subject is a LOPD patient.

Accordingly, in one aspect, the present invention provides a use of the recombinant AAV virus vector of the present invention for driving the expression of a polynucleotide encoding acid alpha-glucosidase (GAA) in a mammalian cell (in particular a human cell), or a use of the recombinant AAV virus vector of the present invention in the preparation of a medicament for driving the expression of a polynucleotide encoding acid alpha-glucosidase (GAA) in a mammalian cell or in one or more tissues or organs in a mammal (especially a human).

Preferably, the medicament is used to express GAA in the heart, liver, muscle, and central nervous system (including the brain and spinal cord) of a mammal.

Preferably, the medicament is administered systemically, e.g. intraperitoneally (i.p.), intramuscularly (i.m.), intraarterially or intravenously (i.v.), preferably intravenously by injection.

In a further aspect, the present invention provides a method for treating a subject having Pompe disease or a subject having acid glucosidase deficiency, and a use of a recombinant AAV vector of the present invention in the preparation of a medicament for the treatment of a subject having Pompe disease or a subject having acid glucosidase deficiency. The treatment comprises administering to the subject any recombinant AAV vector(s) of the present invention, preferably, administering the recombinant AAV vector(s) systemically, such as intraperitoneally (i.p.), intramuscularly (i.m.), intra-arterially, or intraventricularly (i.v.), and preferably via intravenous injection.

In some embodiments of the therapeutic methods and uses according to the present invention, the GAA polypeptide is expressed in the heart, liver, muscle, and central nervous system (including the brain and spinal cord) of the subject after the recombinant AAV vector of the present invention is administered. In yet further embodiments, the administration of the recombinant AAV vector results in an decreased lysosomal glycogen storage in skeletal muscle, cardiac muscle, diaphragm, and central nervous system of the subject, and preferably does not induce or induces low immunogenicity. In some embodiments, the administration of the recombinant AAV vector of the present invention improves cardiac, respiratory and/or skeletal muscle function in the subject. In yet further embodiments, the administration of the recombinant AAV vector of the present invention prevents or alleviates the damages in the central nervous system of the subject, such as the brain, spinal cord and/or neurons, e.g., progressive neurodegeneration resulting from glycogen storage. In some embodiments, the administration of the recombinant AAV vector of the present invention prolongs the survival of the subject.

Accordingly, the present invention also provides the following methods and the use of the recombinant AAV vector of the present invention in the preparation of a medicament for use in the following methods:
(1) Methods for preventing or reducing pathologically excessive lysosomal glycogen storage in cells of subjects in vivo, who have, or are at risk of having, Pompe disease or acid glucosidase deficiency;
(2) Methods for preventing or ameliorating impairment of cardiac, respiratory, and/or skeletal muscle function resulting from excessive lysosomal glycogen storage in subjects who have, or are at risk of having, Pompe disease or acid glucosidase deficiency;
(3) Methods for preventing or alleviating nervous system damage resulting from excessive lysosomal glycogen storage in subjects who have or are at risk of having Pompe disease or acid glucosidase deficiency;
(4) Methods for reducing the burden in the central nervous system resulting from excessive lysosomal glycogen storage and for correcting peripheral organ involvement in subjects who have or are at risk of having Pompe disease or acid glucosidase deficiency;
(5) Methods for prolonging the survival in subjects who have or are at risk of having Pompe disease or acid glucosidase deficiency.

In some embodiments of the therapeutic methods and uses according to the present invention, the recombinant AAV virus vector of the present invention is administered in combination with another therapeutic drug or therapeutic procedure. The another therapeutic agent or therapeutic procedure that can be administered in combination with the recombinant AAV vector of the present invention can be selected from the group consisting of immunomodulators, bronchodilators, acetylcholinesterase inhibitors, respiratory muscle strength training (RMST), enzyme replacement therapy (ERT), and/or diaphragmatic pacing therapy.

### EXAMPLES

The following embodiments in the examples of the present invention will be described clearly and comprehensively. Apparently, the Examples described herein are only a part of embodiments but not all of embodiments of the present invention. Based on the Examples in the present disclosure, all other embodiments, which are obtained by the persons of ordinary skill in the art without creative work, fall within the protection scope of the present invention. Unless otherwise specified, the reagents used in Examples are commercially available.

### Example 1: Construction and Characterization of CAR-Mut Promoters

### 1. Construction of CAR-Mut Promoter

The intron sequence from position 62804 to position 62890 in the human TATA box-binding protein-associated factor 1 gene (GenBank: NG_012771.2) was incorporated at the 3′ end of the CA promoter sequence, which consists of the enhancer sequence of the human CMV virus and the basic promoter of the chicken B-actin protein, and the resulted promoter was named as CAR. The CAR promoter was modified by mutating the T at position 568 at the end of the promoter to a non-T nucleotide to generate the CAR-Mut promoter: CAR-MutC (having T568C mutation, sequence shown in SEQ ID NO: 1), CAR-MutA (having T568A mutation, sequence shown in SEQ ID NO: 2), and CAR-MutG (having T568G mutation, sequence shown in SEQ ID NO: 3).

### 2. Characterization of the Activity of the CAR-Mut Promoter In Vitro

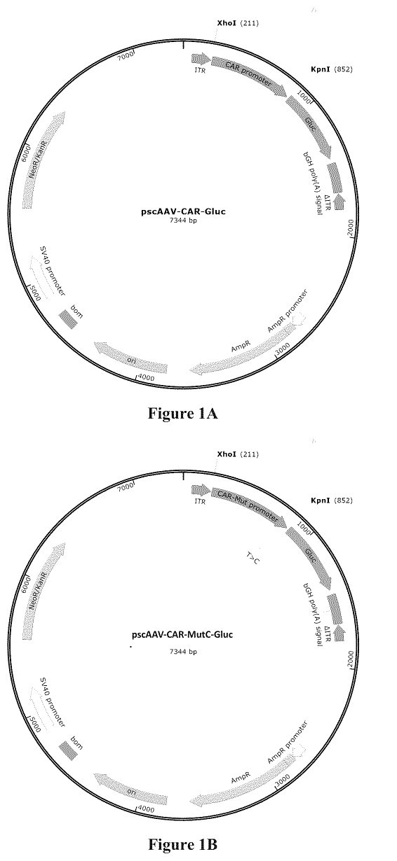

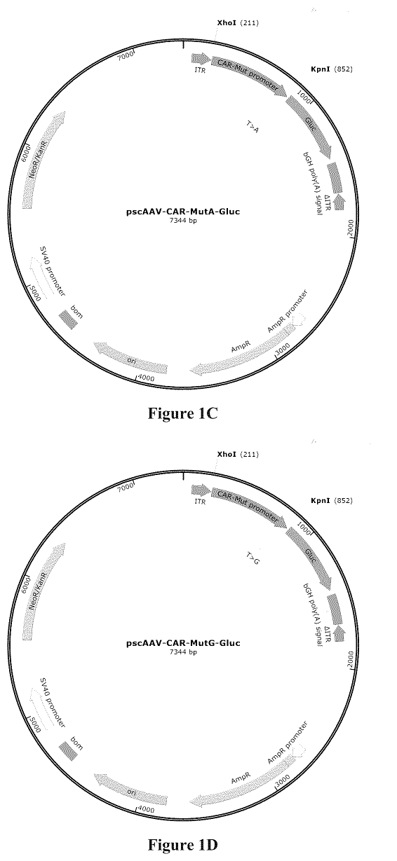

To characterize the promoter CAR-Mut, the pscAAV-CAR-Gluc plasmid vector, shown in FIG. 1A, was constructed, which comprises:
i) ITR derived from the 3′ end of the AAV2 genome (GenBank No. AF043303), with the sequence shown in SEQ ID NO: 5;
ii) CAR promoter, with the sequence shown in SEQ ID NO:4;
iii) Gluc, the nucleotide sequence encoding luciferase reporter gene;
iv) polyadenylation tailing signal from bovine growth hormone, also abbreviated as BGH polyA;
v) AITR, with the sequence shown in SEQ ID NO: 6, which is derived from the 3′ ITR sequence of the AAV2 genome (GenBank No. AF043303) through the deletion of the trs sequence and the D sequence.

The pscAAV-CAR-Gluc plasmid (FIG. 1A) served as the basis, and the CAR promoter in the pscAAV vector was replaced with the CAR-Mut promoter (SEQ ID No. 1, 2 or 3) to obtain the vector pscAAV-CAR-Mut-Gluc (FIG. 1B to FIG. 1D). Briefly, the CAR-Mut promoter sequence was synthesized with XhoI and KpnI restriction sites added at either end. The synthesized sequence was then cloned into pUC57 simple vector (GenScript Biotechnology Co. Ltd., Nanjing) to create pUC57-CAR-Mut. pUC57-CAR-Mut vector and pscAAV-CAR-Gluc vector were respectively double digested with XhoI and KpnI. The CAR-Mut fragment and the pscAAV vector with CAR promoter removed were recovered, ligated and transformed into E. coli DH5α competent cells (Tsingke Biotech Co. Ltd., Beijing). Following screening and verification, pscAAV-CAR-Mut-Gluc, an AAV plasmid vector incorporating the CAR-Mut promoter, was obtained.

Well-growing BHK-21 cells were passaged into a 24-well plate. Upon reaching 60% confluence, pscAAV-CAR-Gluc, pscAAV-CAR-MutC-Gluc, pscAAV-CAR-MutA-Gluc, and pscAAV-CAR-MutG-Gluc were individually transfected into BHK-21 cells in triplicate wells according to the manufacturer's instructions, using Lipofectamine2000 (Invitrogen, USA). After 48 hours post-transfection, 100 μL of supernatant from each well was collected, and the Gluc levels were measured using a Glomax96 microplate luminometer (Promega), and the data were analyzed using the software accompanying with the detector.

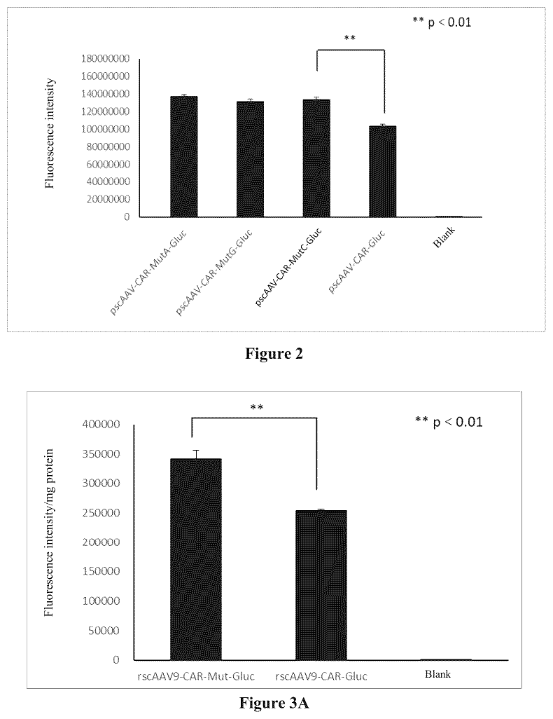

The results (FIG. 2) revealed a substantially significant increase in Gluc expression levels in cells transfected with plasmids pscAAV-CAR-Gluc and pscAAV-CAR-Mut-Gluc compared to the blank control (BHK-21 cells not transfected with a plasmid). In addition, a 29.8% increase was observed with pscAAV-CAR-MutC-Gluc in comparison to pscAAV-CAR-Gluc. No significant differences were observed among pscAAV-CAR-MutA-Gluc, pscAAV-CAR-MutG-Gluc, and pscAAV-CAR-MutC-Gluc.

This indicates an enhanced function following a base substitution at position 568 within the CAR promoter.

### 3. Characterization of the Activity of the CAR-Mut Promoter In Vivo

The functional activity of the recombinant AAV virus comprising the CAR-Mut promoter was evaluated in animals, with the CAR-MutC promoter serving as the representative of the CAR-Mut promoter.

### (1) Preparation of Recombinant AAV Virus

Recombinant viruses were generated by packaging them using a triple-plasmid packaging system, followed by purification to produce the recombinant viruses rscAAV9-CAR-Mut-Gluc and rscAAV9-CAR-Gluc.

Firstly, a plasmid named pAAV-R2C9, which expresses the Rep and Cap proteins of AAV, was constructed. The pAAV-RC plasmid obtained from the AAV Helper Free System (Agilent Technologies, Catalog #240071) served as the basic backbone. The sequence between the HindIII and PmeI restriction sites in the pAAV-RC plasmid was replaced with a synthesized sequence encoding the capsid protein in the AAV9 genome to create the pAAV-R2C9 plasmid, using standard molecular cloning techniques. The pAAV-R2C9 plasmid contained intact versions of the AAV9 cap gene and the AAV2 rep gene, providing the four Rep proteins (Rep78, Rep68, Rep52, and Rep40) and the AAV9 capsid proteins necessary for packaging when recombinant AAV9 viruses were produced through triple-plasmid co-transfection.

The previously constructed AAV vector plasmid (pscAAV-CAR-Gluc vs. pscAAV-CAR-Mut-Gluc), the helper plasmid (pHelper, from AAV Helper Free System, Agilent Technologies), and the plasmid pAAV-R2C9 expressing the Rep and Cap proteins of AAV were thoroughly mixed in a 1:1:1 molar ratio, and then transfected into HEK293 cells using the calcium phosphate method. After 48 hours post-transfection, the cells and culture supernatants were harvested, followed by cesium chloride density gradient centrifugation to isolate and purify the recombinant AAV viruses, resulting in the production of rscAAV9-CAR-Gluc and rscAAV9-CAR-Mut-Gluc.

### (2) Titer Detection of Recombinant AAV Virus

The genomic titers of the prepared recombinant AAV viruses (rAAVs) were determined by dot hybridization. The specific procedure was as follows:

Two primers CAR-Mut-F & CAR-Mut-R were designed in the CAR-Mut promoter:

CAR-Mut-F:
(SEQ ID NO: 8)
5′-GTTCCCATAGTAACGCCAATAGGG-3′
CAR-Mut-R:
(SEQ ID NO: 9)
5′-CCCATAAGGTCATGTACTGGGCAT-3′

PCR was conducted using the CAR-Mut-F and CAR-Mut-R primers to specifically amplify the CAR-Mut promoter, yielding a DNA probe fragment with a length of 175bp. The plasmid pscAAV-CAR-Mut-Gluc and its 2-fold gradient dilution series were used as the standards, while the rAAV samples were diluted in a 2-fold gradient dilution series for testing. These standards and test samples were spotted on a hybridization membrane, which was then subjected to probe hybridization. The detailed procedure can be found in Molecular Cloning, A Laboratory Manual (4th Edition). The titers of the rAAV samples were analyzed and calculated by comparing the hybridization signals of the sample spots with those of a series of standard points using ImigeJ software for grayscale scanning.

### (3) Characterization of In Vivo Functional Activity of Recombinant AAV Virus

Recombinant AAV vectors carrying CAR promoter or CAR-Mut promoter were injected into mice, and then the Gluc levels were assayed to evaluate the functional activity of the promoters. Specifically, a total of eighteen 6-week-old C57 BL/6J wild mice were randomly divided into 3 groups. Group 1 mice were injected with rscAAV9-CAR-Gluc via the tail vein at a dose of 1×10¹³ GC/kg (genome copies/kg). Group 2 mice were injected with rscAAV9-CAR-Mut-Gluc via the tail vein at a dose of 1×10¹³ GC/kg. Group 3 mice were injected with 200 μL PBS via the tail vein as a control. One month post-injection, all mice were euthanized, and the brain tissue, heart, and liver were dissected and isolated from each mouse. Equal amounts of tissue samples were taken, from which total protein was extracted. The concentration of total protein in each group was determined using Pierce BCA Protein Aaasy Kit (ThermoFisher, USA), following the detailed procedure provided in the kit instruction. Gluc levels were measured using a Glomax96 microplate luminometer with 50 μl of protein from each mouse tissue.

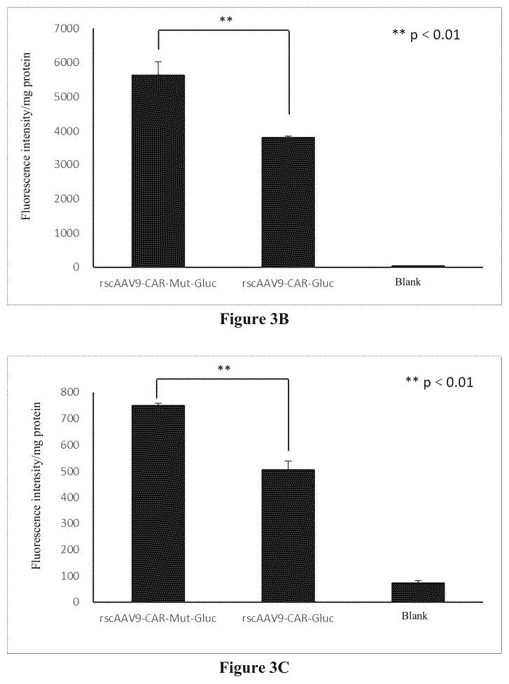

The results (FIGS. 3A, 3B, 3C) showed that after injection of the two AAV vectors, mice efficiently expressed Gluc across various tissues. The Gluc levels in mice injected with rscAAV9-CAR-MutC-Gluc were significantly elevated compared to those injected with rscAAV9-CAR-Gluc in multiple tissues, with a 34.7% increase in the heart (FIG. 3A), a 47.6% increase in the liver (FIG. 3B), and a 48.0% increase in the brain (FIG. 3C).

### Example 2. Construction of Recombinant AAV for Treatment of Pompe Disease

### 1. Construction of AAV Plasmid Vector

In this Example, an AAV plasmid vector was constructed to include a GAA gene of interest along with regulatory elements for expression of the gene and ITR sequences.

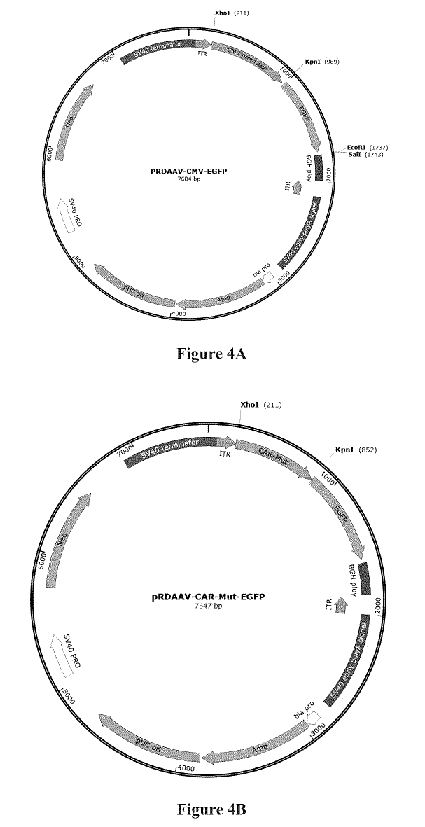

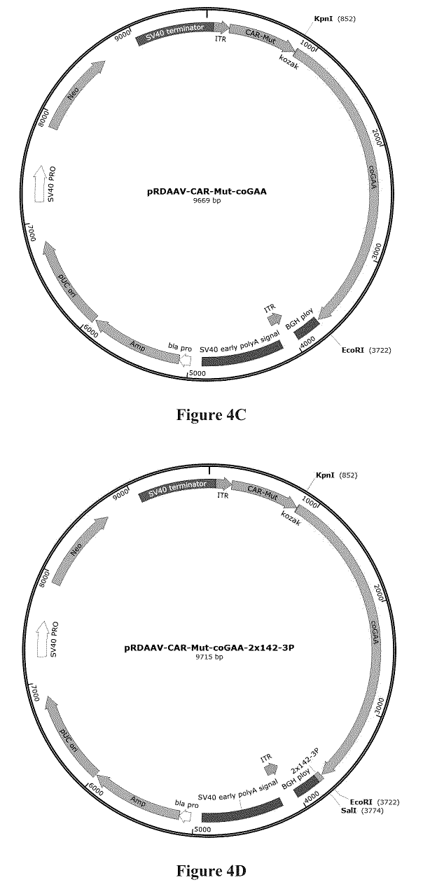

Firstly, using pRDAAV-CMV-EGFP (FIG. 4A) as the basis, pRDAAV-CAR-Mut-EGFP vector (FIG. 4B) was generated by replacing the CMV promoter in the pRDAAV vector with the CAR-MutC promoter (SEQ ID No. 1). The pRDAAV-CMV-EGFP plasmid vector comprises:
i) ITR from AAV2 genome, with the sequence shown in SEQ ID NO:5;
ii) constitutive CMV promoter;
iii) the nucleotide sequence expressing enhanced green fluorescent protein EGFP;
iv) polyadenylation tailing signal from bovine growth hormone, BGH polyA;
v) ITR from AAV2 genome, with the sequence shown in SEQ ID NO: 5.

XhoI and KpnI restriction sites were added at either end of the CAR-MutC promoter sequence (SEQ ID No.1). The sequence having the added restriction sites was synthesized by GenScript Biotechnology Co. Ltd., and then was cloned into pUC57 simple vector (GenScript Biotechnology Co. Ltd., Nanjing) to produce pUC57-CAR-Mut. The vectors pUC57-CAR-Mut and pRDAAV-CMV-EGFP were respectively double digested with XhoI and KpnI. The CAR-Mut promoter fragment and the pRDAAV-CMV-EGFP vector fragment (about 6.9 kb) with the CMV promoter removed were recovered, and the two fragments were ligated together, then transformed into E. coli DH5α competent cells (Tsingke Biotech Co. Ltd., Beijing). Following screening and verification, pRDAAV-CAR-Mut-EGFP (FIG. 4B), an AAV plasmid vector incorporating the CAR-Mut promoter, was obtained.

Next, an artificially synthesized codon-optimized nucleotide sequence for a human GAA (hereinafter abbreviated as coGAA) was cloned into the pRDAAV-CAR-Mut-EGFP vector between the KpnI and EcoRI restriction sites, to obtain pRDAAV-CAR-Mut-coGAA vector (FIG. 4C). Particularly, the codon-optimized cDNA sequence of human GAA gene (coGAA, the sequence shown in SEQ ID No. 10) was synthesized by GenScript Biotechnology Co. Ltd., with KpnI restriction site and Kozak sequence 5′-GCCACC-3′ added upstream of the synthesized coGAA sequence, and TAA stop codon and EcoRI restriction site added downstream of the synthesized coGAA sequence. The synthesized sequence was cloned into pUC57 simple vector (GenScript Biotechnology Co. Ltd., Nanjing) to obtain pUC57-coGAA vector. pUC57-coGAA vector and pRDAAV-CAR-Mut-EGFP vector were respectively double digested with KpnI and EcoRI. The coGAA fragment and the pRDAAV-CAR-Mut-EGFP vector fragment with the EGFP reporter gene removed were recovered, ligated, and then transformed into E. coli DH5α competent cells (Tsingke Biotech Co. Ltd., Beijing). Following screening and verification, the vector pRDAAV-CAR-Mut-coGAA (FIG. 4C) was obtained.

Next, an artificially synthesized miR-142-3 pT fragment (comprising two miR-142-3p target sequences in tandem, see SEQ ID No. 12 for sequence information) was cloned into the vector pRDAAV-CAR-Mut-coGAA between the EcoRI and SalI restriction sites, to obtain pRDAAV-CAR-Mut-coGAA-2×142-3P vector (FIG. 4D). Briefly, oligo primers containing microRNA 142-3 pT were synthesized by Beijing Tsingke Biotech Co. Ltd., and annealed to obtain a 142-3 pT fragment with an upstream EcoRI restriction site and a downstream SalI restriction site. The vector pRDAAV-CAR-Mut-coGAA was linearized by digesting with EcoRI and SalI, and the vector backbone was recovered and ligated with the 142-3 pT fragment, followed by transformation into E. coli DH5α competent cells (Tsingke Biotech Co., Ltd., Beijing). After screening and verification, the vector pRDAAV-CAR-Mut-coGAA-2×142-3P (FIG. 4D) was obtained.

### 2. Preparation and Assay of Recombinant AAV Virus

### (1) Packaging of Recombinant AAV Virus

The Bac-to-AAV system was used for AAV virus packaging. Briefly, the following operations were performed: Sf9 cells were cultivated. Two distinct baculoviruses were prepared via transfection and verified, one encoding the GAA gene of interest and the other encoding AAV-Rep2/Cap9. The two baculoviruses were amplified, Sf9 cells were co-infected with them, and the resulting sf9 cell precipitate was harvested. The cells were lysed to release the AAV viruses, which were purified through ultracentrifugation. The AAV viruses underwent membrane pack-based desalting and concentration, followed by sterile filtration to yield the recombinant AAV virus, rAAV9-CAR-Mut-coGAA-2×142-3P.

The packaging process can be performed by employing methods described in Chen H. Intron Splicing-mediated Expression of AAV Rep and Cap Genes and Production of AAV Vectors in Insect Cells, [J]. Molecular Therapy, the Journal of the American Society of Gene Therapy, 2008, 16(5):924 and patents U.S. Pat. No. 8,945,918 and CN101522903B.

### (2) Titer Assay for Recombinant AAV Virus

The dot hybridization method described in Example 1 was used to determine the genomic titer of the prepared rAAV. The specific procedure was as follows.

PCR was conducted using the CAR-Mut-F (SEQ ID NO: 8) and CAR-Mut-R (SEQ ID NO: 9) primers to specifically amplify the CAR-Mut promoter, yielding a DNA probe fragment with a length of 175 bp. The plasmid pRDAAV-CAR-Mut-coGAA-2×142-3P and its 2-fold gradient dilution series were used as the standards, while the rAAV samples were diluted in a 2-fold gradient dilution series for testing. These standards and test samples were spotted on a hybridization membrane, which was then subjected to probe hybridization. The titers of the rAAV samples were analyzed and calculated using ImigeJ software for grayscale scanning.

### 3. In Vitro Expression of Recombinant AAV Virus for Gene Therapy of Pompe Disease

BHK-21 cells were seeded into 6-well plates. Upon reaching approximately 80% confluency, the cells were detached, counted, and the required amount of viruses was calculated based on a ratio of 50,000 virus particles per cell. The viruses were thoroughly mixed with a fresh medium and added to the corresponding wells in the plates. The plates were incubated at 37° C. After 6-8 hours post-infection, the virus-containing medium was replaced with a fresh medium containing 1% serum. Following an additional 48 hours of incubation, the cells were detached and harvested. Total cellular protein was extracted through repeated freeze-thaw followed by centrifugation. The concentrations of total protein in cells transfected with rAAV9-CAR-Mut-coGAA-2×142-3P and in control cells were determined using the Pierce BCA Protein Assay Kit (ThermoFisher, USA), following the detailed protocol provided in the kit instructions.

After the extraction of total cellular proteins, 10 μl of each of the extracted proteins were taken, and fluorescence was detected (with excitation at 365 nm, and emission at 450 nm) after one-hour reaction under acidic conditions using 4-MUG as a substrate. The concentration of the resulting 4-MU was determined based on a standard curve, which was then utilized to calculate the activity of the GAA enzyme present in the sample. (See, Wenjuan QIU et al., 2010, Establishment and clinical application of a platform for the determination of acid alpha-glucosidase activity in dried blood spots and leukocytes)

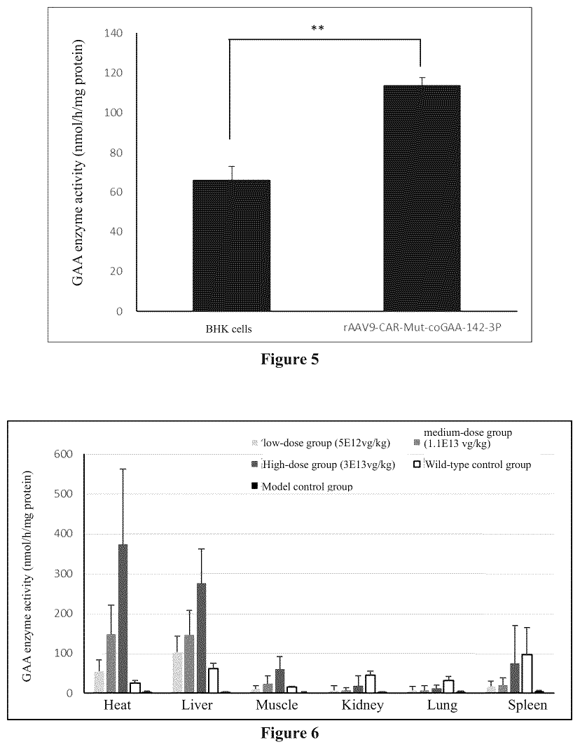

The results are presented in FIG. 5. In BHK-21 cells, the GAA enzyme activity was 65.62±7.49 nmol/h/mg protein. Contrastingly, in BHK-21 cells transfected with the rAAV9-CAR-Mut-coGAA-2×142-3P plasmid, the GAA enzyme activity was 113.60±4.19 nmol/h/mg protein, 1.73 times higher than that in the control cells. (**, p<0.01). This demonstrated that the active GAA protein can be expressed following the transduction of cells with the recombinant AAV for Pompe disease gene therapy.

### Example 3. In Vivo Efficacy Evaluation of Recombinant AAV for Gene Therapy of Pompe Disease in Model Mice

### Experiment 1:

Thirty-two model mice, homozygous for a GAA gene deletion and aged 8-10 weeks (GAA-KO) mice, purchased from Jax lab, No. 004154), were randomly and evenly assigned to four groups. Group I was the model control group, serving as a negative control, with each mouse receiving a single IV injection of 200 μL PBS. The remaining three groups were the low-dose, medium-dose, and high-dose groups, designated as the experimental groups, receiving single IV injections of rAAV9-CAR-Mut-coGAA-2×142-3P at doses of 5E12 vg/kg, 1.1E13 vg/kg, and 3E13 vg/kg, respectively. An additional group consisted of a wild-type control group, which included eight 129 wild mice aged 8-10 weeks for comparison. All mice were euthanized 5 weeks post-injection, and the heart, liver, spleen, lung, kidney, and muscle tissues of each mouse were dissected and isolated.

### GAA Activity Assay

The appropriate amounts of various tissues were collected, and total tissue protein was extracted. Total protein concentration in each group was determined using the Pierce BCA Protein Assay Kit (ThermoFisher, USA), following the kit instructions for the detailed procedure. 5 μl of total protein from each tissue of all mice was used for determination of GAA enzyme activity, with the results presented in FIG. 6.

As depicted in FIG. 6, model mice receiving PBS injections exhibited markedly low enzyme activity due to the absence of GAA protein. In contrast, the groups treated with recombinant AAV showed extensive transduction of peripheral tissues and expression of active GAA protein following a single IV injection of rAAV9-CAR-Mut-coGAA-2×142-3P. Moreover, as the dosage of the injection increased, the enzyme activity in each tissue demonstrated a dose-dependent elevation.

### Histopathological Staining Analysis

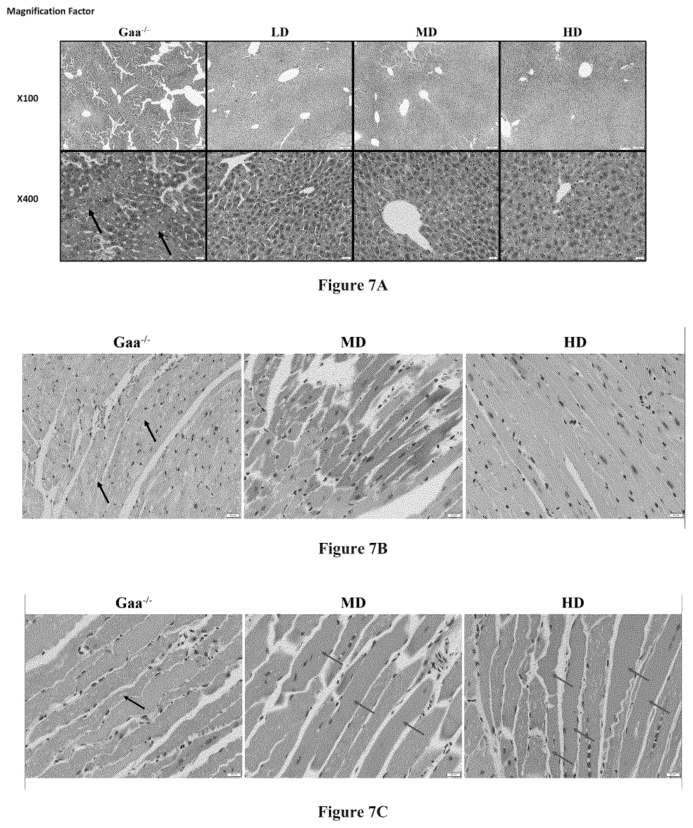

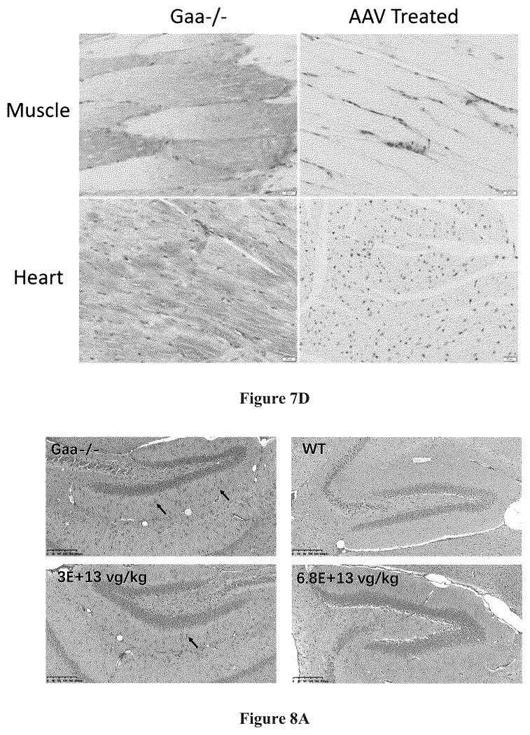

Some of the tissues were collected, cut into appropriate sizes, immersed and fixed in 4% paraformaldehyde, labeled, and sent to Raisedragon's Ltd. for pathology analysis. The results are presented in FIG. 7.

In FIG. 7A, the H&E staining results of liver tissues 5 weeks post intravenous treatment with AAV9-CAR-Mut-coGAA-2×142-3P virus vectors are displayed. In Gaa-/- model mice, extensive multifocal necrosis of the liver (indicated by black arrowheads) was clearly visible, primarily concentrated around the central vein as a consequence of the disease. Following the administration of varying doses (LD, low dose; MD, medium dose; HD, high dose) of AAV9-CAR-Mut-coGAA-2×142-3P, a remarkable reduction in hepatic necrosis area was observed in the model mice. There was no significant karyopyknosis, and a dose-dependent relationship was evident. These results indicate a substantial therapeutic effect of the drug on the liver.

FIG. 7B displays the H&E staining of cardiomyocytes from Gaa-/- model mice post single IV injection. Tissue sections from Gaa-/- model mice treated with PBS revealed a substantial area of vacuole-like degeneration and vascular congestion in the myocardial wall (indicated by black arrows). Cardiomyocytes from Gaa-/- model mice treated with low, medium, and high doses exhibited varying degrees of improvement in a dose-dependent manner. The medium and high doses showed more significant enhancements, with no vacuole-like degeneration of cardiomyocytes or significant vascular congestion in the myocardial wall observed. These results indicate a substantial therapeutic effect of the drug on the cardiomyopathy.

FIG. 7C displays the H&E staining of skeletal muscle cells from Gaa-/- model mice post single IV injection. Tissue sections from Gaa-/- model mice treated with PBS revealed a substantial area of vacuole-like degeneration (indicated by black arrows). Skeletal muscle cells from Gaa-/- model mice treated with low, medium, and high doses exhibited varying degrees of improvement in a dose-dependent manner. The medium and high doses showed more significant enhancements, with no significant inflammatory cell infiltration and the presence of normal myofibers (indicated by red arrow). The most significant effects were observed in the high dose group (FIG. 7C). These results indicate that the drug can significantly ameliorate skeletal muscle injury without the changes induced by toxicity, highlighting the good safety profile of the drug.

FIG. 7D displays the results of PAS staining on skeletal muscle cells and cardiomyocytes of Gaa-/- model mice in exploratory animal model experiments prior to the present experiment. Tissue sections from mice in the PBS-administered group revealed a substantial area of vacuole-like degeneration and intense glycogen staining (upper left and lower left panels). Significant improvements in glycogen accumulation and the restoration of myofiber vacuole-like degeneration were observed in the skeletal and cardiac muscles of Gaa-/- model mice three months post intravenous injection of 5E12 vg/kg of the recombinant AAV (upper right and lower right panels). Thus, consistent with the results presented in FIGS. 7A-7C, the results in FIG. 7D also indicate that the recombinant AAV drug of the present invention can significantly improve skeletal muscle and cardiac muscle injuries, and address the glycogen accumulation pathology underlying Pompe disease.

### Experiment 2

Model mice, homozygous for a GAA gene deletion and aged 8-10 weeks, were treated in a manner essentially similar to that described in Experiment 1. Briefly, the model mice were randomly and evenly assigned into three groups (5 mice per group). Group 1 served as a negative control group, with each mouse receiving a single IV injection of 200 μL PBS. The remaining two groups, designated as the experimental groups, received single IV injections of rAAV9-CAR-Mut-coGAA-2×142-3P at doses of 3E13 vg/kg and 6.8E13 vg/kg, respectively. An additional group was included, which consisted of 129 wild mice aged 8-10 weeks for comparison. All mice were euthanized 5 weeks post-injection, and the cerebral tissue, spinal cord tissue and cerebellar tissue of each mouse were dissected and isolated.

### Histopathological Staining Analysis

Some of the tissues were collected, cut into appropriate sizes, immersed and fixed in 4% paraformaldehyde, labeled, and sent to Raisedragon's Ltd. for pathology analysis.

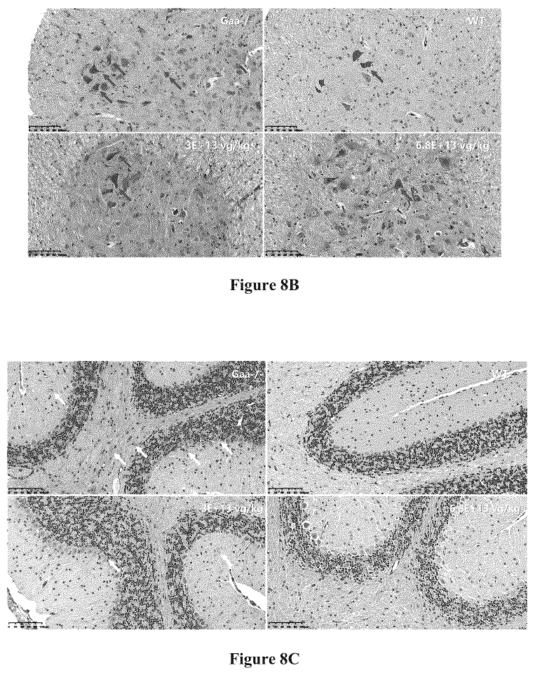

The PAS staining results of cerebral tissues post single intravenous injection of AAV9-CAR-Mut-coGAA-2×142-3P are depicted in FIG. 8A. The results revealed extensive glycogen accumulation in glial cells of Pompe disease model animals, leading to a substantial area of positive PAS staining (upper left panel, black arrow), a phenomenon absent in wild-type mice (upper right panel). With increasing doses of the recombinant AAV drug, Pompe disease model mice exhibited effective mitigation of glial cell glycogen accumulation, with no detectable glycogen accumulation in glial cells at a dose of 6.8E+13 vg/kg (lower left and lower right panels).

The PAS staining results of spinal cord tissues post single intravenous injection of AAV9-CAR-Mut-coGAA-2×142-3P are depicted in FIG. 8B. The results revealed a higher presence of PAS glycogen strongly positive cells in the anterior horn of the spinal cord in Pompe disease model animals, with a relatively high proportion of PAS-positive neurons (upper left panel), indicating glycogen accumulation in the spinal cord of the model mice. This is consistent with the report in the literatures regarding model mice. Only a few motor neurons in the anterior horn of wild-type mice exhibited strong glycogen positivity (upper right panel), suggesting occasional cells with robust glycogen metabolism among motor neurons in the anterior horn. Following recombinant AAV treatment, the proportion of PAS-positive neurons in the 3E+13 vg/kg dose group exhibited small changes. While the dose was increased to 6.8E+13 vg/kg, there was a remarkable decrease in strongly PAS-positive motor neurons in the spinal cord, with only a few neurons PAS-glycogen positive, resembling the WT characteristics (lower left and lower right panels).

The PAS staining results of cerebellar tissue post single intravenous injection of AAV9-CAR-Mut-coGAA-2×142-3P are depicted in FIG. 8C. The results revealed insignificant glycogen accumulation in cerebellar Purkinje cells of Pompe disease model animals. However, there was a more pronounced accumulation of glycogen granules in glial cells surrounding Purkinje cells, with significant glycogen accumulation in the cerebellar medullary region (upper left panel, white arrow). Following recombinant AAV administration, a decreasing trend in the number of glycogen-positive cells around Purkinje cells in cerebellar tissue was observed at the 3E+13 vg/kg dose (lower left panel). No glycogen accumulation was detected when the dose was increased to 6.8E+13 vg/kg (lower right panel).

The results of FIGS. 8A-8C demonstrate the recombinant AAV drug of the present invention effectively ameliorates the pathological changes in the nervous system caused by the disease, including cerebral, spinal cord, and cerebellar tissues. This indicates that the recombinant AAV drug has the ability to dose-dependently clear glycogen from the central nervous system post intravenous injection, and the ability to traverse the blood-brain barrier and correct intracellular glycogen metabolism disorders.

### GAA Activity Assay

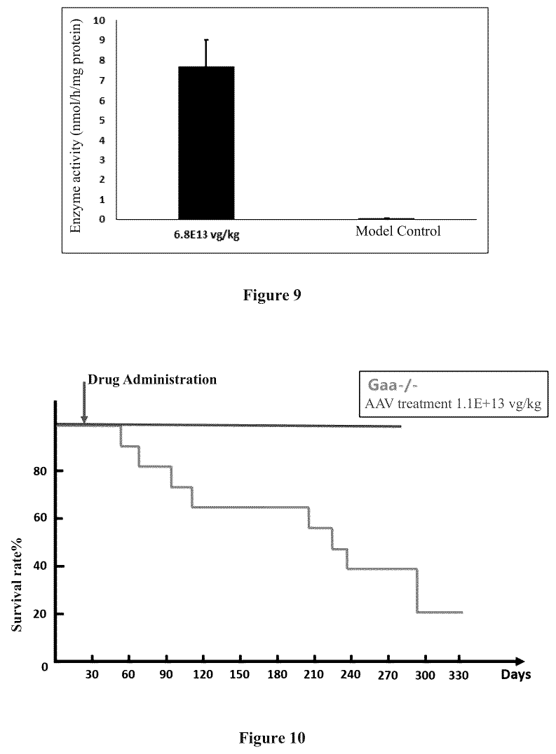

Total tissue protein was extracted from appropriate amounts of various tissues. The protein concentration for each group was determined using the Pierce BCA Protein Assay Kit (ThermoFisher, USA) following the kit instructions for the detailed procedure. 5 μl of total protein from each of the tissues of all mice was utilized to determine GAA enzyme activity, with the results presented in FIG. 9.

As depicted in FIG. 9, the 6.8E+13 vg/kg dose group exhibited a significant increase in GAA enzyme activity levels in brain tissues. Pompe model mice was GAA enzyme activity negative in the brain tissues. Following a single intravenous injection of AAV9-CAR-Mut-coGAA-2×142-3P, the GAA enzyme activity in brain tissue was found to be 7-8 times higher than that of the model control (n=5, p<0.001). This indicates the recombinant AAV virus has the ability to traverse the blood-brain barrier via the blood system, deliver the GAA expression vector to the central nervous system, and effectively express the active GAA enzyme. Thus, the recombinant AAV drug of the present invention has a targeted corrective impact on improving the enzyme deficiency characteristic within the central nervous system in Pompe disease.

### Experiment 3

A control recombinant AAV9 virus lacking the miRNA-142 target sequence, AAV9-CAR-Mut-coGAA, was constructed and compared with the recombinant AAV9 virus containing the miRNA-142 target sequence, AAV9-CAR-Mut-coGAA-2×142-3P, in terms of therapeutic effectiveness and serum antibody titer.

Substantially as described in Examples 1 and 2, the therapeutic impact was assessed following the administration of the recombinant AAV9 virus.

The serum anti-drug antibody titers were determined as follows: five weeks post-AAV administration, mice were euthanized, and blood samples were collected. Serum separation was followed by ELISA to detect the anti-GAA antibody titers in the mouse serum samples.

The results indicated that the presence or absence of the miRNA-142 target sequence did not result in a significant difference in therapeutic efficacy. However, the recombinant AAV9 virus incorporating the miRNA-142 target sequence exhibited a reduction in anti-drug antibody titers post IV administration. The antibody titer was 1:800 in the recombinant AAV virus experiment group having the miRNA-142 target sequence, whereas the antibody titer exceeded 1:6400 in the control group lacking the miRNA-142 target sequence. This suggests that the inclusion of the miRNA-142 target sequence reduces the level of drug-related inhibitors and facilitates the induction of immune tolerance.

### Experiment 4

Sixteen model mice, homozygous for a GAA gene deletion and aged 8-10 weeks (GAA-KO mice, purchased from Jax lab), were randomly and evenly assigned to two groups. Group 1 served as a negative control, with each mouse receiving a single intravenous injection of 200 μL PBS. The other group served as the experimental group, receiving a single intravenous injection of rAAV9-CAR-Mut-coGAA-2×142-3P at a dosage of 1.1E13 vg/kg. The survival was monitored, and the survival curves were documented. These results are presented in FIG. 10.

As depicted in FIG. 10, following treatment of the Gaa-/- model mice with 1.1E+13 vg/kg, there was a significant improvement in the natural disease progression of Pompe model mice. The median survival time was notably prolonged compared to that of the model animals, and a 100% survival rate was observed in the treatment group. These results signify that the drug demonstrated the anticipated therapeutic effects, eliminating the disease's impact and aligning the survival rates of the model mice with those of wild-type normal mice.

The illustrative embodiments of the present invention have been detailed above. One skilled in the art can refer to the information provided herein to implement the invention of this application by adapting the process parameters. It is important to note that various similar substituents and modifications will readily be obvious to persons skilled in the art, and shall be considered to fall within the scope of the present invention. The methods and uses of this invention have been described through preferred examples, allowing those skilled in the art to make alterations or appropriate modifications and combinations to the methods and uses described herein, without departing from the content, spirit, and scope of the present invention, to achieve and utilize the technology of this invention.

1. A mutant promoter comprising a polynucleotide selected from the group consisting of:
(i) a polynucleotide of SEQ ID NO: 4.
(ii) a polynucleotide having at least 95%, 96%, 97%, 98%, 99%, 99.5% identity to SEQ ID NO: 4.
(iii) a polynucleotide obtained by substitution, deletion or addition of one or several nucleotides to the polynucleotide of SEQ ID NO: 4,
and wherein the polynucleotide has a mutation at positions 562-572 of SEQ ID NO:4 or at positions corresponding thereto, preferably said mutation is a mutation from T to C or G or A at position 568 or at a corresponding position, and more preferably a T to C mutation;
preferably, the mutant promoter increases the expression of a gene of interest functionally linked thereto, e.g., increases by 1%-70%, such as at least 5%, at least 10%, at least 20%, at least 30%, at least 40%, or at least 50%, at least 60%, relative to a reference promoter consisting of a corresponding polynucleotide without the mutation;
more preferably, the mutant promoter is a constitutive promoter, wherein the mutant promoter increases the expression of the gene of interest in mammalian cells or tissues relative to the reference promoter, e.g., increases the expression of the gene of interest in mammalian peripheral tissues and/or central nervous tissues, e.g., increases the expression in mammalian tissues selected from the group consisting of the heart, the liver and/or the brain, and preferably the mammal is a human or a non-human mammal.

2. The promoter of claim 1, wherein the promoter comprises a nucleotide sequence selected from any one of SEQ ID NOs: 1 to 3, or a nucleotide sequence differing therefrom by one or several nucleotide substitutions, deletions and/or additions and having equivalent promoter activity;
preferably, the promoter comprises or consists of the nucleotide sequence of SEQ ID NO: 1.

3. An expression construct comprising the following elements functionally linked to each other in a transcriptional direction:
the promoter of any one of claim 1 or 2,
a polynucleotide sequence encoding acid alpha-glucosidase (GAA), preferably a codon-optimized sequence encoding a human GAA polypeptide, more preferably a sequence of SEQ ID NO:10.

4. The expression construct of claim 3, further comprising at least one (e.g., 2 to 6, preferably 2) immune-related miRNA target sequence located after the coding nucleic acid sequence, in particular a miR-142 target sequence, such as a miR-142 target sequence comprising at least one (e.g., 2 or 4) copies of the SEQ ID NO: 11 sequence, e.g., a sequence shown in SEQ ID NO: 12;
optionally, the expression construct further comprises one or more selected from the group consisting of the following:
(1) a Kozak sequence located before the coding nucleic acid sequence,
(2) a transcription terminator, such as a polyA signaling sequence, preferably selected from the group consisting of a SV40 late polyA sequence, a rabbit β-globin polyA sequence, and a bovine growth hormone poly A sequence, more preferably a bovine growth hormone poly A sequence.

5. The expression construct of claim 4, wherein the expression construct further comprises 5′ adeno-associated virus inverted terminal repeat (ITR) sequence located upstream of the promoter, and 3′ adeno-associated virus inverted terminal repeat (ITR) sequence located downstream of the transcriptional terminator, preferably the ITR sequences are wild-type ITR sequences, or one of the ITRs is a wild-type ITR sequence and the other of the ITRs is AITR sequence lacking a functional terminal resolution site (trs) and optionally a D sequence.

6. A vector comprising the expression construct of any one of claims 3-5, wherein the vector is a plasmid or a virus vector, such as a recombinant AAV virus vector or a baculovirus vector.

7. A recombinant adeno-associated virus (AAV) vector, wherein the genome of the recombinant AAV vector comprises:
a. 5′ and 3′ AAV inverted terminal repeat (ITR) sequences, and
b. an expression construct located between the 5′ and 3′ ITRs, wherein the expression construct comprises the following elements functionally linked to each other in a transcriptional direction:
the promoter of any one of claim 1 or 2,
optionally, a Kozak sequence,
a polynucleotide encoding a human acid alpha-glucosidase (GAA),
optionally, at least one (e.g., 2 to 6) immune-related miRNA target sequence, in particular a miR-142 target sequence, e.g., a miR-142-3p target sequence comprising at least one (e.g., 2 or 4) copies of the SEQ ID NO: 11 sequence,
a transcription terminator, such as a polyA signaling sequence, preferably selected from the group consisting of a SV40 late polyA sequence, a rabbit β-globin polyA sequence, and a bovine growth hormone polyA sequence, more preferably a bovine growth hormone polyA sequence.

8. The recombinant AAV virus vector of claim 7, wherein the polynucleotide encoding GAA is codon-optimized for human, and more preferably, the polynucleotide comprises the sequence of SEQ ID NO: 10.

9. The recombinant AAV virus vector of claim 7 or 8, wherein the ITRs are wild-type AAV2 ITR sequences, or one of the ITRs is an AA2 AITR sequence lacking a functional terminal resolution site (trs) and optionally a D sequence.

10. The recombinant AAV virus vector of any one of claims 7-9, wherein the vector is an ssAAV vector or an scAAV vector.

11. The AAV virus vector of any one of claims 7-10, wherein the recombinant AAV vector comprises a capsid protein from AAV9 serotype, preferably, the recombinant AAV vector is AAV2/9 vector.

12. A host cell comprising the promoter of any one of claims 1-2 or the expression construct of any one of claims 3-5 or the vector of claim 6.

13. The use of the recombinant AAV virus vector of claims 7-11 for driving the expression of a polynucleotide encoding acid alpha-glucosidase (GAA) in a mammalian cell, or in the manufacture of a medicament for driving the expression of a polynucleotide encoding acid alpha-glucosidase (GAA) in a mammalian cell, or in one or more tissues or organs in a mammal;
preferably, the medicament is used to express the GAA in the heart, liver, muscle, and central nervous system (including the brain and spinal cord) of the mammal;
preferably, the medicament is administered systemically, e.g. intraperitoneally (i.p.), intramuscularly (i.m.), intra-arterially or intravenously (i.v.) by injection, preferably intravenously by injection.

14. A method for prevention or treatment of a subject having Pompe disease or a subject having acid glucosidase deficiency, comprising administering to the subject the recombinant AAV vector of claims 7-11;
preferably, the recombinant AAV vector is administered systemically, e.g. intraperitoneally (i.p.), intramuscularly (i.m.), intra-arterially or intravenously (i.v.) by injection, preferably intravenously by injection.

15. The method of claim 14, wherein the recombinant AAV vector is administered to increase expression of the GAA polypeptide in peripheral tissues (preferably, heart, liver, muscle) and the central nervous system (including the brain and the spinal cord) of the subject.

16. The method of claim 14, wherein the administration of the recombinant AAV vector reduces the lysosomal glycogen storage in peripheral tissues (preferably, heart, liver, muscle) and the central nervous system (including the brain and the spinal cord, preferably glial cells) of the subject, and preferably ameliorates the tissue damages resulting from the glycogen storage, and preferably with no or low immunogenicity induced.

17. The method of claim 14, wherein the recombinant AAV virus vector is administered in combination with another agent, preferably the agent is a recombinant GAA protein for enzyme replacement therapy (ERT).

18. A pharmaceutical composition comprising the vector of claim 6 or the recombinant AAV virus vector of any one of claims 7-11 and a pharmaceutically acceptable carrier.

19. A cell comprising: (i) a first vector encoding one or more rep proteins of adeno-associated virus and/or one or more cap proteins of adeno-associated virus; and (ii) a second vector comprising the expression construct of any one of claims 3-5;
preferably, the first vector is a plasmid and the second vector is a plasmid; the cell is a mammalian cell, optionally wherein the mammalian cell is a HEK293 cell; or
preferably, the first vector is a baculovirus vector and the second vector is a baculovirus vector; the cell is an insect cell, optionally wherein the insect cell is a sf9 cell.

20. A method for producing the recombinant AAV virus vector of any one of claims 7-11, wherein the method comprises the steps of:
(i) providing the cell of claim 19;
(ii) culturing the cell under conditions that permit packaging of the recombinant AAV; and
(iii) harvesting the cultured host cell or the culture medium to collect the recombinant AAV virus vector.


SEQUENCE LISTING (13 sequences)

SEQ ID NO: 1 - DNA, 631 bp, artificial
  synthetic
attgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattacccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgacgcgctatgcggccgcgtgagtgagtgatttgatctaaacgcctttttgtaatcaagtgactgtgtgtgtgtgtatctgagtgcctgattcttttcatcacag

SEQ ID NO: 2 - DNA, 631 bp, artificial
  synthetic
attgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattacccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgacgcgctatgcggccgcgtgagtgagtgatttgatctaaaagcctttttgtaatcaagtgactgtgtgtgtgtgtatctgagtgcctgattcttttcatcacag

SEQ ID NO: 3 - DNA, 631 bp, artificial
  synthetic
attgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattacccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgacgcgctatgcggccgcgtgagtgagtgatttgatctaaaggcctttttgtaatcaagtgactgtgtgtgtgtgtatctgagtgcctgattcttttcatcacag

SEQ ID NO: 4 - DNA, 631 bp, artificial
  synthetic
attgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattacccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgacgcgctatgcggccgcgtgagtgagtgatttgatctaaatgcctttttgtaatcaagtgactgtgtgtgtgtgtatctgagtgcctgattcttttcatcacag

SEQ ID NO: 5 - DNA, 145 bp, artificial
  synthetic
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 6 - DNA, 121 bp, artificial
  synthetic
ccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 7 - DNA, 230 bp, artificial
  synthetic
gcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctat

SEQ ID NO: 8 - DNA, 24 bp, artificial
  synthetic
gttcccatagtaacgccaataggg

SEQ ID NO: 9 - DNA, 24 bp, artificial
  synthetic
cccataaggtcatgtactgggcat

SEQ ID NO: 10 - DNA, 2868 bp, artificial
  synthetic
gccaccatgggcgtgcggcacccaccttgctctcacagactgctggccgtgtgcgcactggtgagcctggcaaccgccgccctgctgggccacatcctgctgcacgacttcctgctggtgcccagggagctgtccggaagctccccagtgctggaggagacccacccagcacaccagcagggcgcctctaggccaggcccccgcgatgcacaggcacacccaggcaggccccgcgccgtgccaacccagtgcgacgtgccacccaacagcaggtttgactgtgcccccgataaggccatcacacaggagcagtgcgaggcaaggggatgctgttatatccctgcaaagcagggactgcagggagcacagatgggacagccatggtgtttctttcctccatcttaccccagctataagctggagaatctgtctagctccgagatgggctacacagccaccctgacaagaaccacaccaacattctttcccaaggatatcctgaccctgaggctggacgtgatgatggagacagagaaccgcctgcacttcaccatcaaggatcccgccaatcggagatatgaggtgcctctggagaccccacacgtgcactctagggcccctagcccactgtactccgtggagttctctgaggagccatttggcgtgatcgtgaggcgccagctggatggccgcgtgctgctgaacaccacagtggcccccctgttctttgccgaccagttcctgcagctgagcacatccctgccctcccagtatatcaccggcctggccgagcacctgtctcctctgatgctgtctaccagctggacacggatcaccctgtggaacagagacctggcaccaacccctggagcaaatctgtacggcagccaccccttctatctggccctggaggatggaggatccgcccacggcgtgtttctgctgaactctaatgccatggacgtggtgctgcagccaagccccgccctgtcctggaggtctaccggcggcatcctggacgtgtacatcttcctgggccctgagccaaagtccgtggtgcagcagtacctggacgtggtgggctatcctttcatgcccccttactggggactgggatttcacctgtgcagatggggctattctagcacagccatcaccaggcaggtggtggagaacatgacccgcgcccactttccactggatgtgcagtggaatgacctggattacatggactcccggagagacttcaccttcaacaaggacggcttccgggattttccagcaatggtgcaggagctgcaccagggcggcaggcgctacatgatgatcgtggatcctgcaatctcctctagcggacctgcaggaagctacagaccatatgacgagggcctgcggagaggcgtgttcatcacaaacgagaccggccagcctctgatcggcaaggtctggccaggctccaccgccttcccagacttcaccaatccaaccgccctggcatggtgggaggacatggtggccgagttccacgaccaggtgccttttgatggcatgtggatcgacatgaacgagccatctaatttcatcaggggaagcgaggacggatgcccaaacaatgagctggagaatccaccatatgtgcctggagtggtgggaggcacactgcaggcagcaaccatctgtgcctcctctcaccagtttctgtctacacactataacctgcacaatctgtacggactgaccgaggcaatcgcaagccacagagccctggtgaaggcacggggcacaagacctttcgtgatctcccggtctacctttgccggacacggcagatacgcaggacactggaccggcgacgtgtggagctcctgggagcagctggcctctagcgtgccagagatcctgcagttcaacctgctgggcgtgcccctggtgggagcagacgtgtgcggctttctgggcaatacaagcgaggagctgtgcgtgcggtggacccagctgggagccttctatccctttatgcgcaaccacaatagcctgctgtccctgcctcaggagccatacagcttctccgagcctgcccagcaggccatgaggaaggccctgacactgcgctatgccctgctgccacacctgtacaccctgtttcaccaggcacacgtggcaggagagacagtggccaggcccctgttcctggagtttcctaaggattcctctacctggacagtggaccaccagctgctgtggggagaggccctgctgatcacccccgtgctgcaggcaggcaaggcagaggtgacaggctatttccctctgggcacatggtacgatctgcagaccgtgccagtggaggccctgggaagcctgcctccaccacctgcagcaccaagggagcctgccatccactccgagggacagtgggtgacactgccagcacctctggacaccatcaacgtgcacctgcgggccggctatatcatcccactgcagggacctggactgaccacaaccgagagcagacagcagccaatggcactggcagtggccctgaccaagggaggagaggcaaggggagagctgttctgggacgatggcgagtccctggaggtgctggagagaggcgcctacacacaggtcatcttcctggcccggaacaatacaatcgtgaatgagctggtgagagtgacctctgagggagcaggactgcagctgcagaaggtgacagtgctgggagtggcaaccgcaccacagcaggtgctgtccaacggcgtgcccgtgagcaatttcacatactcccctgataccaaggtgctggacatctgcgtgagcctgctgatgggcgagcagtttctggtgtcctggtgttagtaa

SEQ ID NO: 11 - DNA, 23 bp, artificial
  synthetic
tccataaagtaggaaacactaca

SEQ ID NO: 12 - DNA, 46 bp, artificial
  synthetic
tccataaagtaggaaacactacatccataaagtaggaaacactaca

SEQ ID NO: 13 - protein, 952 aa, human
MGVRHPPCSHRLLAVCALVSLATAALLGHILLHDFLLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC